(19) United States
(12) Patent Application Publication
(10) Pub. No.: US 2017/0200291 A1
(43) Pub. Date: Jul. 13, 2017

(54) MAGNETIC RESONANCE IMAGING APPARATUS AND IMAGE RECONSTRUCTION METHOD

(71) Applicant: HITACHI, LTD., Tokyo (JP)

(72) Inventors: Yasuhiro KAMADA, Tokyo (JP); Katsunari NAGASHIMA, Tokyo (JP)

(73) Assignee: HITACHI, LTD., Tokyo (JP)

(21) Appl. No.: 15/320,564

(22) PCT Filed: Jul. 8, 2015
(86) PCT No.: PCT/JP2015/069645

§ 371 Date: Dec. 20, 2016

(30) Foreign Application Priority Data
Jul. 29, 2014 (JP) 2014-153818

(51) Int. Cl. G06T 11/00; G06T 5/10; G01R 33/36; G01R 33/48; A61B 5/055; A61B 5/00

(52) U.S. Cl. CPC G06T 11/003; A61B 5/055; A61B 5/742; A61B 5/7257; G01R 33/3621; G01R 33/4818; G06T 5/10; G06T 2210/41; G06T 2207/10088

(57) ABSTRACT

In k space parallel imaging, the image reconstruction processing is increased in speed without deteriorating the image quality. Therefore, interpolation processing in the image reconstruction processing of the k space parallel imaging is segmented into element data generation processing in which measured k space data of one of channels is used such that element data of interpolation data of all of the channels is generated, and addition processing in which the generated element data is added for each channel. The element data generation processing is segmented into units set in advance, for example, for each channel and the element data generation processing is executed in parallel.

### TECHNICAL FIELD

The present invention relates to a technology of magnetic resonance imaging and particularly relates to a technology of parallel imaging in which a reception coil having multiple channels is used.

### BACKGROUND ART

An MRI apparatus is an apparatus which measures a nuclear magnetic resonance (NMR) signal generated in an object, particularly in a nucleus spin of atoms configuring the tissue of a human body and two-dimensionally or three-dimensionally performs image forming of a form or a function of the head, the abdomen, the limbs, or the like. In video recording, phase encoding and frequency encoding which vary depending on a gradient magnetic field is applied to the NMR signal. The measured NMR signal is subjected to two-dimensional or three-dimensional Fourier transform and is reconstructed as an image. Hereinafter, a space in which measured signal data is disposed will be referred to as a k space, data disposed in the k space will be referred to as k space data, and a space obtained by performing Fourier transform of the k space will be referred to as an image space.

An MRI technique includes parallel imaging in which an RF reception coil (hereinafter, reception coil) configured to have at least two reception channels is used and the phase encoding (in a case of three-dimensional measurement, the phase encoding and/or slice encoding) is thinned to R multiplication and measured such that a video recording time is shortened to 1/R multiplication.

In a thinned and measured k space data, even if Fourier transform is performed without any change, aliasing occurs and image forming cannot be correctly performed. As an image reconstruction technique for solving the problem, there is a technique in which cyclical properties of the k space are utilized and the thinned k space data is restored through interpolation (for example, refer to PTL 1 and PTL 2). The technique is called k space parallel imaging.

In the k space parallel imaging, unmeasured data of the k space (hereinafter, will be referred to as the k space of the reception channel) having the signal data acquired in each reception channel disposed therein is restored through the interpolation, and performs compositing of (channel compositing) of each piece of the k space data after restoration. The interpolation and the restoration of the k space data of each reception channel require the k space data of all of the reception channels. Therefore, an image reconstruction time in the k space parallel imaging extends so as to be proportional to the square of the number of reception channels.

As a method of increasing the image reconstruction processing of the k space parallel imaging in speed, there is an image space method (for example, refer to PTL 3). The image space method is a technique in which interpolation processing in the k space is transformed into processing in the image space such that a convolution computation is omitted. In the image space method, the interpolation processing in the k space is expressed as the convolution computation of the thinned k space data and an interpolation kernel and becomes multiplication processing of an aliasing image and an aliasing elimination map by performing Fourier transform of both the elements. In this technology, since a computation space is merely transformed from the k space to the image space, the processing result becomes the same as that of the k space parallel imaging in the related art.

### CITATION LIST

### Patent Literature

PTL 1: Specification of U.S. Pat. No. 7,282,917

PTL 2: Specification of U.S. Pat. No. 6,841,998

PTL 3: Specification of U.S. Pat. No. 7,279,895

### SUMMARY OF INVENTION

### Technical Problem

In respect to the aspect of SNR and performance of parallel imaging, the number of reception channels tends to increase as years go by. Therefore, reconstruction processing of k space parallel imaging is required to be increased in speed.

Generally, in order to increase the speed of computation processing, parallel processing is often adopted. However, in the k space parallel imaging, as described above, in order to perform processing of signal data of one reception channel, the signal data of all of the reception channels is used. Therefore, even if the processing is subjected to paralleling for each channel, pieces of data used between computations contend with each other, thereby internally resulting in serial processing. Thus, consequentially, the computation speed is not improved.

According to an image space method, a computation of each reception channel is increased in speed approximately several times. However, a portion for extension caused due to an increase of the number of reception channels becomes significant. As a result, the computation is required to be further increased in speed.

The present invention has been made in consideration of the aforementioned circumstances, and an object thereof is to provide a technology in which image reconstruction processing is increased in speed without deteriorating the image quality in the k space parallel imaging.

### Solution to Problem

According to the present invention, interpolation processing in image reconstruction processing of k space parallel imaging is segmented into element data generation processing in which measured k space data of one of channels is used such that element data of interpolation data of all of the channels is generated, and addition processing in which the generated element data is added for each channel. The element data generation processing is segmented into units set in advance, for example, for each channel and the element data generation processing is executed in parallel.

### Advantageous Effects of Invention

According to the present invention, in the k space parallel imaging, the image reconstruction processing can be increased in speed without deteriorating the image quality.

### BRIEF DESCRIPTION OF DRAWINGS

FIG. 1 is a configuration diagram of a magnetic resonance imaging apparatus of a first embodiment.

FIG. 2 is a functional block diagram of a control system of the first embodiment.

FIGS. 3(a) to 3(c) are diagrams for describing interpolation processing of k space parallel imaging.

FIGS. 4(a) and 4 (b) are diagrams for describing interpolation coefficient calculation processing of the k space parallel imaging.

FIG. 5 is a diagram for describing interpolation processing in the related art.

FIG. 6 is a flow chart of image reconstruction processing of k space parallel imaging in the related art.

FIGS. 7(a) to 7(e) are diagrams for describing interpolation processing of the first embodiment.

FIGS. 8(a) and 8(b) are diagrams describing the interpolation processing of the k space parallel imaging.

FIG. 9(a) is a diagram for describing interpolation processing in the related art, and FIG. 9(b) is a diagram for describing the interpolation processing of the first embodiment.

FIG. 10 is a flow chart of the image reconstruction processing performed based on k space parallel imaging of the first embodiment.

FIG. 11 is a flow chart of image reconstruction processing performed based on k space parallel imaging of Modification Example 1 of the first embodiment.

FIG. 12 is a flow chart of image reconstruction processing of an image space method in the related art.

FIG. 13 is a flow chart of image reconstruction processing of an image space method of a second embodiment.

FIG. 14 is a flow chart of image reconstruction processing performed based on parallel imaging of a third embodiment.

### DESCRIPTION OF EMBODIMENTS

### First Embodiment

Hereinafter, a first embodiment to which the present invention is applied will be described with reference to the drawings. In all of the drawings describing each embodiment, repetitive description of the elements having the same function in the elements having the same name and the same reference sign will be omitted.

[Configuration of MRI Apparatus]

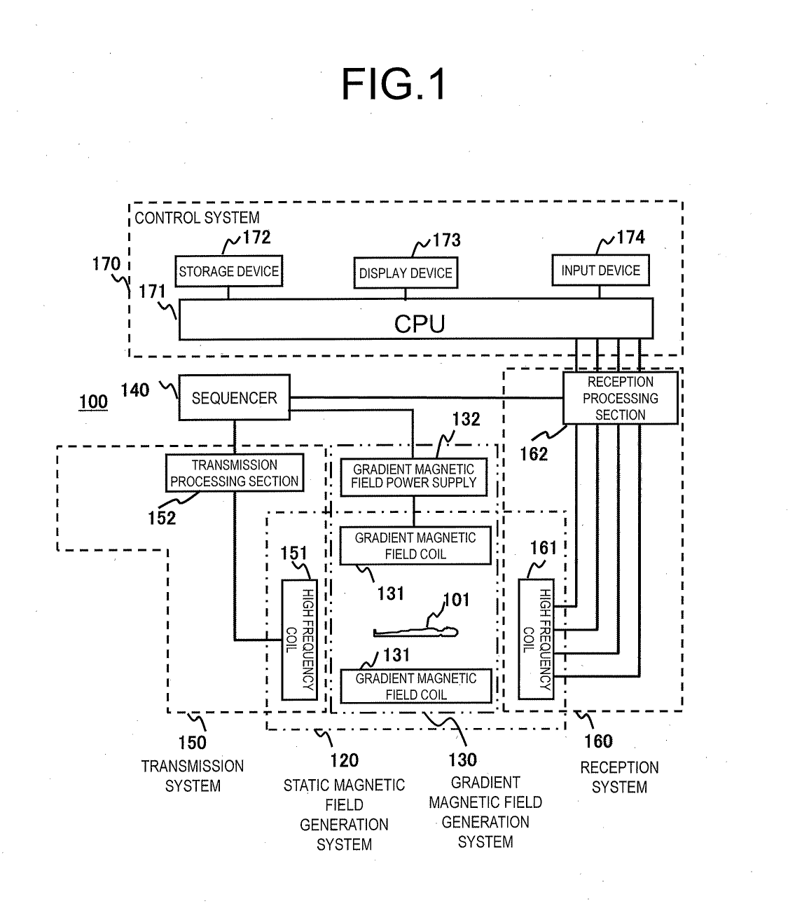

First, a general overview of an example of an MRI apparatus of the present embodiment will be described. FIG. 1 is a block diagram illustrating the overall configuration of an MRI apparatus 100 of the present embodiment. The MRI apparatus 100 of the present embodiment obtains a tomographic image of an object by utilizing an NMR phenomenon. As illustrated in FIG. 1, the MRI apparatus 100 includes a static magnetic field generation system 120, a gradient magnetic field generation system 130, a transmission system 150, a reception system 160, a control system 170, and a sequencer 140.

In a case of a vertical magnetic field method, the static magnetic field generation system 120 generates a uniform static magnetic field in a direction orthogonal to the body axis of an object 101 in the space around the object 101, and in a case of a horizontal magnetic field method, the static magnetic field generation system 120 generates a uniform static magnetic field in a direction of the body axis. The static magnetic field generation system 120 is provided with a static magnetic field generation source which is disposed around the object 101 and adopts a permanent magnet method, a normal conduction method, or a super-conduction method.

The gradient magnetic field generation system 130 is provided with gradient magnetic field coils 131 wound around directions of three axes X, Y, and Z, that is, the coordinate system (apparatus coordinate system) of the MRI apparatus 100, and a gradient magnetic field power supply 132 driving each of the gradient magnetic field coils. The gradient magnetic field generation system 130 drives the gradient magnetic field power supply 132 for each of the gradient magnetic field coils 131 in response to a command from the sequencer 140, thereby applying gradient magnetic fields Gx, Gy, and Gz in the directions of three axes X, Y, and Z.

The transmission system 150 emits a high frequency magnetic field pulse (hereinafter, will be referred to as “RF pulse”) to the object 101 in order to cause nuclear magnetic resonance in an atomic nucleus spin of atoms configuring biological tissue of the object 101. The transmission system 150 is provided with a transmission processing section 152 including a high frequency oscillator (synthesizer), a modulator, and a high frequency amplifier; and a high frequency coil (transmission coil) 151 on a transmission side. The high frequency oscillator generates an RF pulse and outputs the RF pulse at the timing based on a command from the sequencer 140. The modulator performs amplitude modulation with respect to the output RF pulse. The high frequency amplifier amplifies the RF pulse which has been subjected to amplitude modulation, thereby supplying the amplified RF pulse to the transmission coil 151 disposed near the object 101. The transmission coil 151 emits the supplied RF pulse to the object 101.

The reception system 160 detects a nuclear magnetic resonance signal (echo signal, NMR signal) radiated due to nuclear magnetic resonance of a nucleus spin configuring atoms of biological tissue of the object 101. The reception system 160 is provided with a high frequency coil (reception coil) 161 on a reception side; and a reception processing section 162 including a synthesizer, an amplifier, a quadrature phase detector, and an A/D converter.

The reception coil 161 is disposed near the object 101 and detects a responding NMR signal (reception signal) of the object 101 induced by an electromagnetic wave emitted from the transmission coil 151, in each channel. In the present embodiment, the reception coil 161 is a multi-channel coil provided with multiple reception channels (hereinafter, will be simply referred to as channels). A reception signal of each channel is amplified in the reception processing section 162, is detected at the timing based on a command from the sequencer 140, is converted into a digital quantity, and is sent to the control system 170 for each channel.

FIG. 1 illustrates an example of a case where there are four channels. Each of the channels has a serial number so as to be identifiable.

The sequencer 140 repetitively applies RE′ pulses and gradient magnetic field pulses in response to a predetermined pulse sequence. A high frequency magnetic field, a gradient magnetic field, signal receiving timing, and strength are recorded in the pulse sequence, which is held in the control system 170 in advance. The sequencer 140 operates in response to an instruction from the control system 170 and transmits various types of command required in collecting data of a tomographic image of the object 101 to the transmission system 150, the gradient magnetic field generation system 130, and the reception system 160.

The control system 170 controls operations of the MRI, apparatus 100 in its entirety, performs signal processing, conducts various types of computation such as image reconstruction, and displays and retains processing results. The control system 170 is provided with a CPU 171, a storage device 172, a display device 173, and an input device 174. The storage device 172 is configured with an internal storage device such as a hard disk, and an external storage device such as an external hard disk, an optical disk, and a magnetic disk. The display device 173 is a display device such as CRT and liquid crystal. The input device 174 is an interface for inputting various types of control information of the MRI apparatus 100 and control information of processing performed in the control system 170. For example, the input device 174 is provided with a track ball or a mouse, and a keyboard. The input device 174 is disposed near the display device 173. While watching the display device 173, an operator inputs instructions and data required in various types of processing of the MRI apparatus 100 interactively through the input device 174.

The CPU 171 executes a program held in the storage device 172 in advance in response to an instruction input by the operator, thereby realizing each process of the processing and each function of the control system 170, such as controlling operations of the MRI apparatus 100 and processing of various types of data. For example, when the data from the reception system 160 is input to the control system 170, the CPU 171 executes processing such as the signal processing and the image reconstruction. Then, as a result thereof, a tomogram of the object 101 is displayed through the display device 173 and is stored in the storage device 172.

In the present embodiment, as described below, the processing is increased in speed through paralleling. In order to realize the increase in speed, the control system 170 of the present embodiment is configured to be able to perform parallel processing. For example, the control system 170 is provided with multiple CPUs 171 which can operate in parallel. In addition, the CPU 171 may be configured with a multi-core CPU which can operate in parallel. Otherwise, as the CPU 171, multiple processing substrates may be provided.

All or a portion of the functions of the control system 170 may be realized through hardware such as an application specific integrated circuit (ASIC) and a field-programmable gate array (FPGA). In addition, various types of data used in the processing of the functions, and various types of data generated during the processing are stored in the storage device 172.

In a case of the vertical magnetic field method, the transmission coil 151 and the gradient magnetic field coils 131 are installed so as to face the object 101 within a space of a static magnetic field of the static magnetic field generation system 120 in which the object 101 is inserted, and in a case of the horizontal magnetic field method, the transmission coil 151 and the gradient magnetic field coils 131 are installed so as to surround the object 101. In addition, the reception coil 161 is installed so as to face or to surround the object 101.

Presently, a clinically spread nuclear species of an imaging target for an MRI apparatus is a hydrogen nucleus (proton) which is a main configuration substance of the object 101. In the MRI apparatus 100, information related to spatial distribution of the proton density and spatial distribution of relaxation times in an excitation state is image-formed, and a form or a function of the head, the abdomen, the limbs, or the like of a human body is imaged two-dimensionally or three-dimensionally.

[Functional Configuration of Control System]

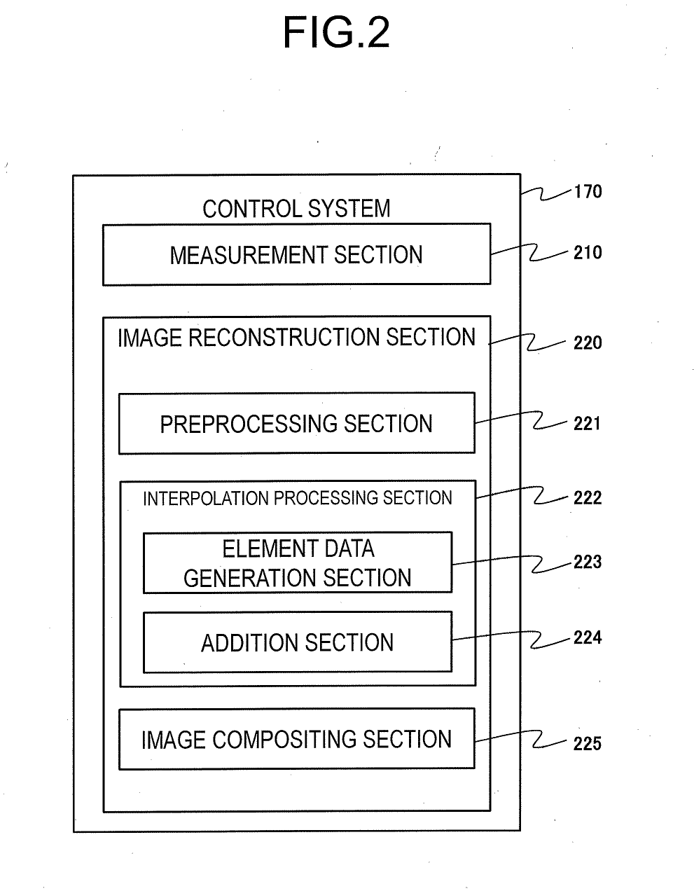

In the present embodiment, based on an echo signal acquired in the multiple channels, an image is reconstructed through k space parallel imaging. In order to realize the reconstruction, as illustrated in FIG. 2, the control system 170 of the present embodiment is provided with a measurement section 210 and an image reconstruction section 220.

[Measurement Section]

The measurement section 210 performs thinning of an encoding step of a k space and measures k space data for each channel. When the measurement is performed, in order to calculate a coefficient (hereinafter, interpolation coefficient) to be used in data interpolation, a low range portion of the k space is measured more densely than a high range portion. Hereinafter, the k space data of each channel is on the premise that the low range portion is densely measured without thinning the encoding step and the high range portion other than the low range portion is thinned and measured.

[Image Reconstruction Section]

The image reconstruction section 220 obtains a reconstruction image by applying a computation based on the k space parallel imaging utilizing cyclical properties of the k space to the measured k space data. In a k space parallel imaging method, data of a position of the thinned k space of each channel is generated as interpolation data by using the measured k space data of all of the channels, and the k space data is restored. An image for each channel is reconstructed based on the restored k space data of each channel and the reconstruction image is obtained by performing compositing of the reconstructed images. In addition, when the interpolation data is generated, the interpolation coefficient is used. The processing in which the interpolation data is generated by using the interpolation coefficient and the thinned k space data is restored will be referred to as interpolation processing.

Therefore, as illustrated in FIG. 2, the image reconstruction section 220 of the present embodiment is provided with a preprocessing section 221 which calculates the interpolation coefficient used in computation, by using the measured k space data, an interpolation processing section 222 which executes the interpolation processing in which the calculated interpolation coefficient is applied to the measured k space data and generates a channel image which is an image for each channel, and an image compositing section 225 which performs compositing of the channel images and obtains the reconstruction image.

The interpolation processing of the present embodiment is processing in which interpolation data that is the data of a position of the thinned k space is generated by using the measured k space data. When the interpolation data is generated, the interpolation coefficient is used. Therefore, the preprocessing section 221 calculates the interpolation coefficient used in the interpolation processing based on the measured k space data. The calculation of the interpolation coefficient will be described later in detail. In addition, the image compositing section 225 performs compositing of the channels through a technique of sum-of-square compositing, for example.

[Interpolation Processing]

Before describing the interpolation processing section 222 of the present embodiment in detail, an overview of the interpolation processing performed through the k space parallel imaging will be described. As described above, in the k space parallel imaging, the data of a position of one thinned k space is interpolated by using the k space data at a position adjacent to the position thereof in the k spaces of all of the channels.

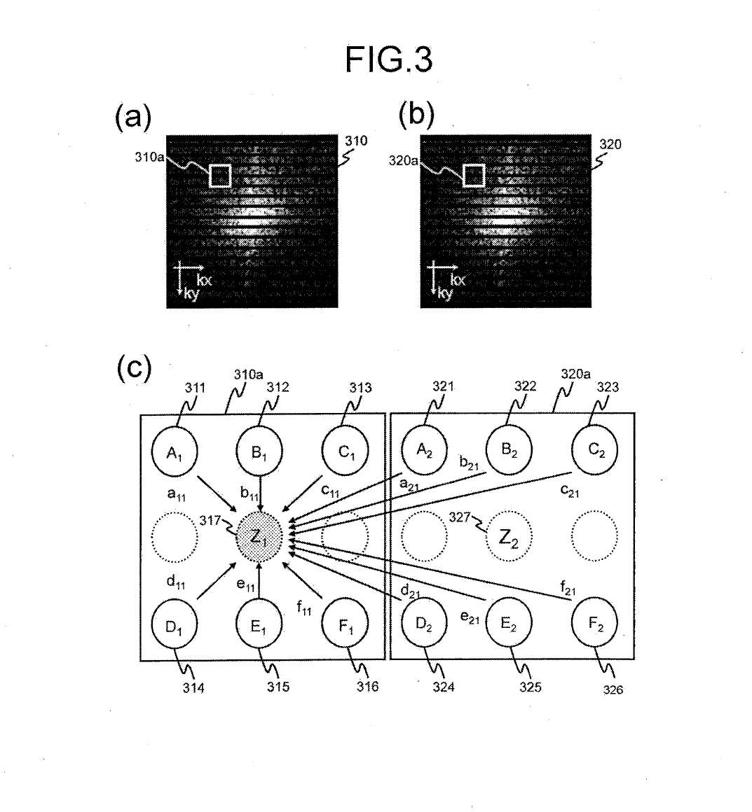

An example of a case where there are two channels will be specifically described. FIGS. 3(a) to 3(c) are diagrams for describing an overview of the k space parallel imaging in a case where there are two channels.

FIG. 3(a) illustrates k space data 310, that is k space data in which signal data acquired in a channel 1 (Ch#1) is disposed, and FIG. 3 (b) illustrates k space data 320, that is k space data in which signal data acquired in a channel 2 (Ch#2) is disposed. The pieces of data are thinned and acquired.

FIG. 3(c) schematically illustrates an enlarged small region 310a in the k space data 310 including pixels 311 to 316 and 317, and an enlarged small region 320a in the k space data 320 including pixels 321 to 326 and 327.

Here, description will be given with reference to an example of a case where complex data of the pixel 317 is generated within the k space data 310a through interpolation using complex data of a group of adjacent pixels including six pixels 311 to 316, that is, a case where six pieces of data are used in total including three pieces in frequency encoding directions and two pieces in phase encoding directions per channel in order to interpolate the k space data of the pixel 317.

The pixels 311 to 316 and the pixels 321 to 326 are pieces of k space data which are actually measured. The pieces of complex data thereof are respectively referred to as A₁ to F₁ and A₂ to F₂. The pixel 317 is the k space data generated through interpolation. In addition, the pixels 311 and 321, 312 and 322, 313 and 323, 314 and 324, 315 and 325, and 316 and 326 are pixels at the same pixel position.

In the k space parallel imaging, the pixel 317 of the k space of the channel 1 is calculated in accordance with the following Expression (1) by using pixel values (k space data) of the group of adjacent pixels of all of the channels (channel 1 and channel 2).

Z₁=a₁₁×A₁+b₁₁×B₁+c₁₁×C₁

+d₁₁×D₁+e₁₁×E₁+f₁₁×F1

+a₂₁×A₂+b₂₁×B₂+C₂₁×C₂

+d₂₁×D₂+e₂₁×E₂+f₂₁×F₂ (1)

Here, a₁₁ to f₁₁, and a₂₁ to f₂₁ are respectively the interpolation coefficients.

In addition, similarly, the pixel 327 of the channel 2 is calculated in accordance with the following Expression (2).

Z₂=a₁₂×A₁+b₁₂×B₁+c₁₂×C₁

+d₁₂×D₁+e₁₂×E₁+f₁₂×F₁

+a₂₂×A₂+b₂₂×B₂+C₂₂×C₂

+d₂₂×D₂+e₂₂×E₂+f₂₂×F₂ (2)

Here, a₁₂ to f₁₂, and a₂₂ to f₂₂ are respectively the interpolation coefficients.

Hereinafter, in this specification, the k space data used in generation of the interpolation data will be referred to as interpolation origin data. The k space in which the interpolation data is present will be referred to as an interpolation destination k space or an interpolation destination channel, and the k space in which the interpolation origin data is present will be referred to as an interpolation origin k space or an interpolation origin channel.

[Calculation of Interpolation Coefficient]

The interpolation coefficient used when the interpolation data is calculated is obtained by extracting low range data of the k space and calculating the extracted result. Generally, the region to be extracted has approximately ±16 encodes in both the frequency encoding direction and the phase encoding direction.

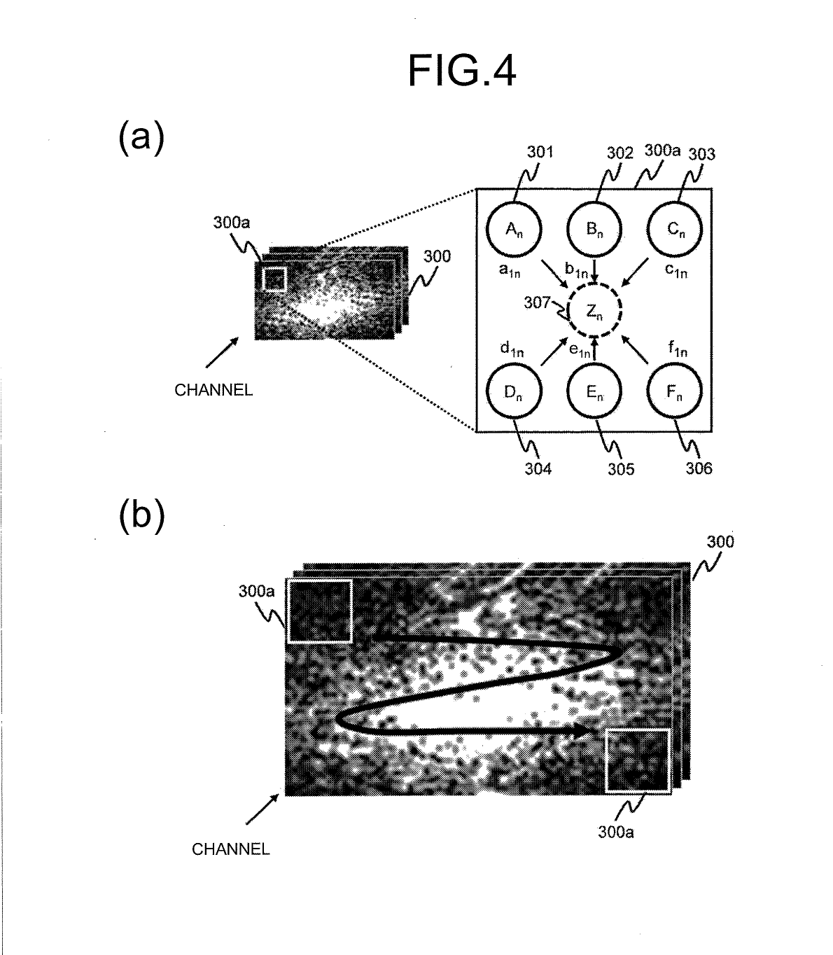

A technique of calculating the interpolation coefficient will be described with reference to FIGS. 4(a) and 4 (b). In the diagrams, the reference sign 300 indicates k space low range data of one channel. The reference sign 300a indicates the k space data which is enlarged while focusing on a predetermined pixel 307 and the group of adjacent pixels 301 to 306 in the k space low range data 300.

As described above, the interpolation coefficient (complex number) is a coefficient by which the complex data of each pixel is multiplied when the complex data of the pixel 307 (interpolation destination f pixel) is calculated based on the complex data of the adjacent pixels 301 to 306 (interpolation origin pixel). Here, the number of all of the channels is N (N is an integer equal to 1 or greater. There is no theoretical upper limit of N. However, the practical upper limit is approximately 1,028), and description will be given with reference to an example of a case where the complex data of a channel n (n is an integer ranging from 1 to N) is calculated. The pieces of the complex data of the group of the adjacent pixels 301 to 306 of the channel n are respectively Aₙ to Fₙ, and the interpolation coefficients (complex numbers) used for calculating the complex data of the channel n are respectively a₁ₙ to f_{Nn}.

The complex data Zₙ (the subscript indicates the channel number) of the pixel 307 (interpolation destination pixel) of the channel n is expressed through the following Expression (3) by using each piece of the complex data A₁ to F₁, and so on to A_{N} to F_{N} (the subscript indicates the channel number) of the group of the adjacent pixels 301 to 306 (group of interpolation origin pixels) of each channel.

$$\begin{matrix}{Z}_{n} = {a}_{1 n} \cdot {A}_{1} + {b}_{1 n} \cdot {B}_{1} + ⋯ + {f}_{1 n} \cdot {F}_{1} + \\ {a}_{2 n} \cdot {A}_{2} + {b}_{2 n} \cdot {B}_{2} + ⋯ + {f}_{2 n} \cdot {F}_{2} + \\ ⋮ \\ {a}_{Nn} \cdot {A}_{N} + {b}_{Nn} \cdot {B}_{N} + ⋯ + {f}_{Nn} \cdot {F}_{N}\end{matrix} \qquad ( 3 )$$

As described above, since the extracted k space low range data is densely measured, the pieces of the complex data A₁ to F₁, so on to A_{N} to F_{N}, and Zₙ of each pixel are measured data. In a case of performing interpolation by using the complex data of six pixels adjacent to each other, since there are 6×N unknown interpolation coefficients, the above-referenced Expression (3) is prepared with 6×N pixels different from each other, and the expression is solved as a simultaneous equation, thereby obtaining each interpolation coefficient.

For example, there are P pixels in the k space low range data 300. The factor P is an integer equal to or greater than 6×N. As illustrated in FIG. 4(b), k space pixel numbers p (p is an integer ranging from 1 to P) are respectively applied to all of the pixels in the k space low range data 300. The complex data of the pixel 307 having the pixel number p in the channel n is Zₙ(P), and each piece of the complex data of the group of the adjacent pixels 301 to 306 are respectively Aₙ(p) to Fₙ(p).

By using thereof, regarding the k space pixel numbers to P, an expression similar to the above-referenced Expression (3) is prepared (hereinafter, Expression (4)). The P expressions are prepared.

$$\{ \begin{matrix}{Z}_{n} ( 1 ) = {a}_{1 n} \cdot {A}_{1} ( 1 ) + {b}_{1 n} \cdot {B}_{1} ( 1 ) + ⋯ + {f}_{Nn} \cdot {F}_{N} ( 1 ) \\ {Z}_{n} ( 2 ) = {a}_{1 n} \cdot {A}_{1} ( 2 ) + {b}_{1 n} \cdot {B}_{1} ( 2 ) + ⋯ + {f}_{Nn} \cdot {F}_{N} ( 2 ) \\ ⋮ \\ {Z}_{n} ( P ) = {a}_{1 n} \cdot {A}_{1} ( P ) + {b}_{1 n} \cdot {B}_{1} ( 1 ) + ⋯ + {f}_{Nn} \cdot {F}_{N} ( P )\end{matrix} \qquad ( 4 )$$

When being expressed in a matrix, the following Expression (5) is established.

$$[ \begin{matrix}{Z}_{n} ( 1 ) \\ {Z}_{n} ( 2 ) \\ ⋮ \\ {Z}_{n} ( P )\end{matrix} ] = [ \begin{matrix}{A}_{1} ( 1 ) & {B}_{1} ( 1 ) & ⋯ & {F}_{N} ( 1 ) \\ {A}_{1} ( 2 ) & {B}_{1} ( 2 ) & ⋯ & {F}_{N} ( 2 ) \\ ⋮ & ⋮ & ⋮ & ⋮ \\ {A}_{1} ( P ) & {B}_{1} ( P ) & ⋯ & {F}_{N} ( P )\end{matrix} ] [ \begin{matrix}{a}_{1 n} \\ {b}_{1 n} \\ ⋮ \\ {f}_{Nn}\end{matrix} ] \qquad ( 5 )$$

Here, the elements of the above-referenced Expression (5) are respectively expressed as a vector Z, a matrix A, and a vector X, and the following Expression (6) is established.

$$Z = AX \\ Here , \\ Z = [ \begin{matrix}{Z}_{n} ( 1 ) \\ {Z}_{n} ( 2 ) \\ ⋮ \\ {Z}_{n} ( P )\end{matrix} ] , A = [ \begin{matrix}{A}_{1} ( 1 ) & {B}_{1} ( 1 ) & ⋯ & {F}_{N} ( 1 ) \\ {A}_{1} ( 2 ) & {B}_{1} ( 2 ) & ⋯ & {F}_{N} ( 2 ) \\ ⋮ & ⋮ & ⋮ & ⋮ \\ {A}_{1} ( P ) & {B}_{1} ( P ) & ⋯ & {F}_{N} ( P )\end{matrix} ] , X = [ \begin{matrix}{a}_{1 n} \\ {b}_{1 n} \\ ⋮ \\ {f}_{Nn}\end{matrix} ] \qquad ( 6 )$$

An unknown matrix X configured with the interpolation coefficients can be solved by changing Expression (6) to the following Expressions (7) and (8).

A^{H}Z=A^{H}AX (7)

X=(A^{H}A)⁻¹A^{H}Z (8)

The factor H indicates a conjugate transposition matrix. By obtaining X, the interpolation coefficient for calculating the complex data of the channel n is obtained.

The interpolation coefficient is generated for each interpolation destination channel in each interpolation origin channel. Thus, hereinafter, in this specification, the interpolation coefficient calculated through the above-referenced technique is defined by expressing through the following Expression (9).

cₘₙ[i][j] (9)

Here, the factor c indicates the interpolation coefficient (complex number), the factor m indicates the interpolation origin channel number, the factor n indicates the interpolation destination channel number, and the factors i and j indicate relative positions (kx-direction and ky-direction) based on interpolation target data, respectively. For simplification, limitations of −1≦i≦1 and −1≦j≦1 are applied. In addition, the factors m and n are integers respectively satisfying 1≦m≦N, 1≦n≦N, and the factor N indicates the number of all of the channels (integer).

That is, the interpolation coefficient expressed through the above-referenced Expression (9) is the k space data obtained by acquiring the k space data of the pixels at the positions (kx and ky) in the channel m, in the k space data acquired in the nth channel (hereinafter, the channel n). The interpolation coefficient thereof is an interpolation coefficient used when performing interpolation by using a data group of pixels (kx+i and ky+j) which are respectively separated as much as i in the kx-direction and as much as j in the ky-direction.

As described above, in the k space parallel imaging of the present embodiment, in order to interpolate one piece of the k space data, six pieces of the interpolation origin data in total, that is, three pieces of the interpolation origin data in the frequency encoding direction per channel, and two pieces of the interpolation origin data in the phase encoding direction per channel are used. Therefore, when there are N channels, 6×N² interpolation coefficients per image are calculated.

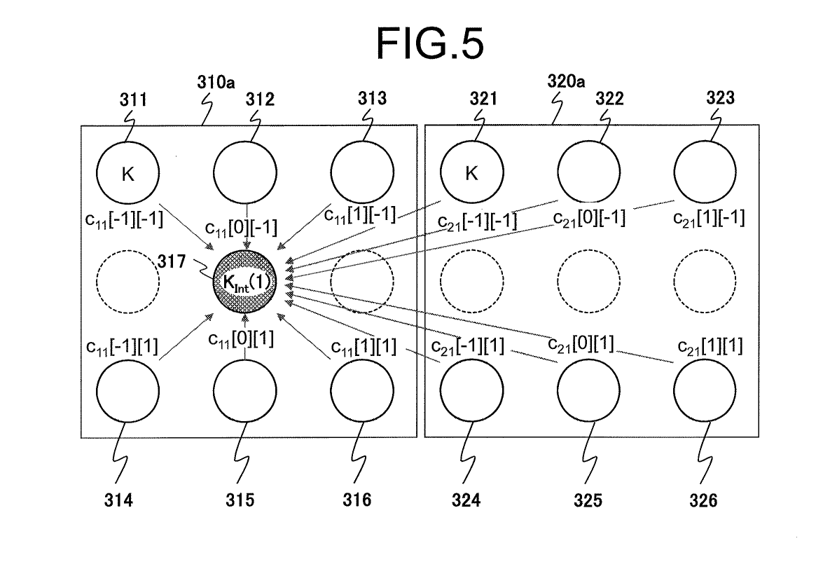

When FIG. 3(c) is expressed by using the expression, FIG. 5 is obtained. Here, the channel 1 and the channel 2 are the interpolation origin channels, and the channel 1 is the interpolation destination channel. Similar to that above, the factor cₘₙ[i][j] indicates the interpolation coefficient, the factor m indicates the interpolation origin channel number, the factor n indicates the interpolation destination channel number, and the factors i and j indicate the relative positions (kx-direction and ky-direction) based on the interpolation target data. In addition, the data (interpolation data) generated through interpolation is defined as K_{Int}(n). The factor n indicates the interpolation destination channel number.

As illustrated in FIG. 5, when the k space data of the pixel 317 of the channel 1 is generated as interpolation data K_{Int}(1), the interpolation origin data of the channel 1 and the interpolation origin data of the channel 2 are used. In this case, the interpolation coefficients of C₁₁[−1][−1] to C₁₁[1][1] are applied to the data of each of the pixels 311 to 316 which are the interpolation origin data of the channel 1, and the interpolation coefficients of C₂₁[−1][−1] to C₂₁[1][1] are applied to the data of each of the pixels 321 to 326 which are the interpolation origin data of the channel 2.

When this is expressed in an expression, the following Expression (10) is established.

K_{Int}(1,kx,ky)

=c₁₁[−1][−1]×K(1,kx−1,ky−1)+c₁₁[0][−1]×K(1,kx,ky−1)+c₁₁[1][−1]×K(1,kx+1,ky−1)

+c₁₁[−1][1]×K(1,kx−1,ky+1)+c₁₁[0][1]×K(1,kx,ky+1)+c₁₁[1][1]×K(1,kx+1,ky+1)

+c₁₁[−1][−1]×K(2,kx−1,ky−1)+c₂₁[0][−1]×K(2,kx,ky−1)+c₂₁[1][−1]×K(2,kx+1,ky−1)

+c₂₁[−1][1]×K(2,kx−1,ky+1)+c₂₁[0][1]×K(2,kx,ky+1)+c₂₁[1][1]×K(2,kx+1,ky+1) (10)

Here, the factors n, kx, and ky indicate the coordinates of the interpolation data (channel number, frequency encoding position, and phase encoding position), the factor K_{Int}(n, kx, and ky) indicates the interpolation data, and the factor K (1 to N, kx−1 to kx+1, and ky−1 to ky+1) indicates the k space data (interpolation origin data) used in interpolation, respectively.

[Flow of Image Reconstruction Processing Using Interpolation Processing in Related Art]

In this manner, in the interpolation processing performed through the k space parallel imaging method, in order to restore the k space which is thinned and measured, regarding all of the thinned pixels of all of the channels, the interpolation destination data K_{Int} is calculated. In this case, as is clear from the above-referenced Expression (10), in the interpolation processing performed through the k space parallel imaging method, in order to obtain the pixel value (interpolation data) of a predetermined interpolation destination pixel of the channel n, the pixel value (interpolation origin data) of the adjacent pixel adjacent to the interpolation destination pixel in all of the channels is required.

Therefore, in the interpolation processing performed through the technique in the related art, the interpolation origin data of all of the interpolation origin channels are used, and the processing in which the interpolation data is obtained by calculating the above-referenced Expression (10) is repeated in order for each interpolation destination channel as much as all of pixels requiring interpolation.

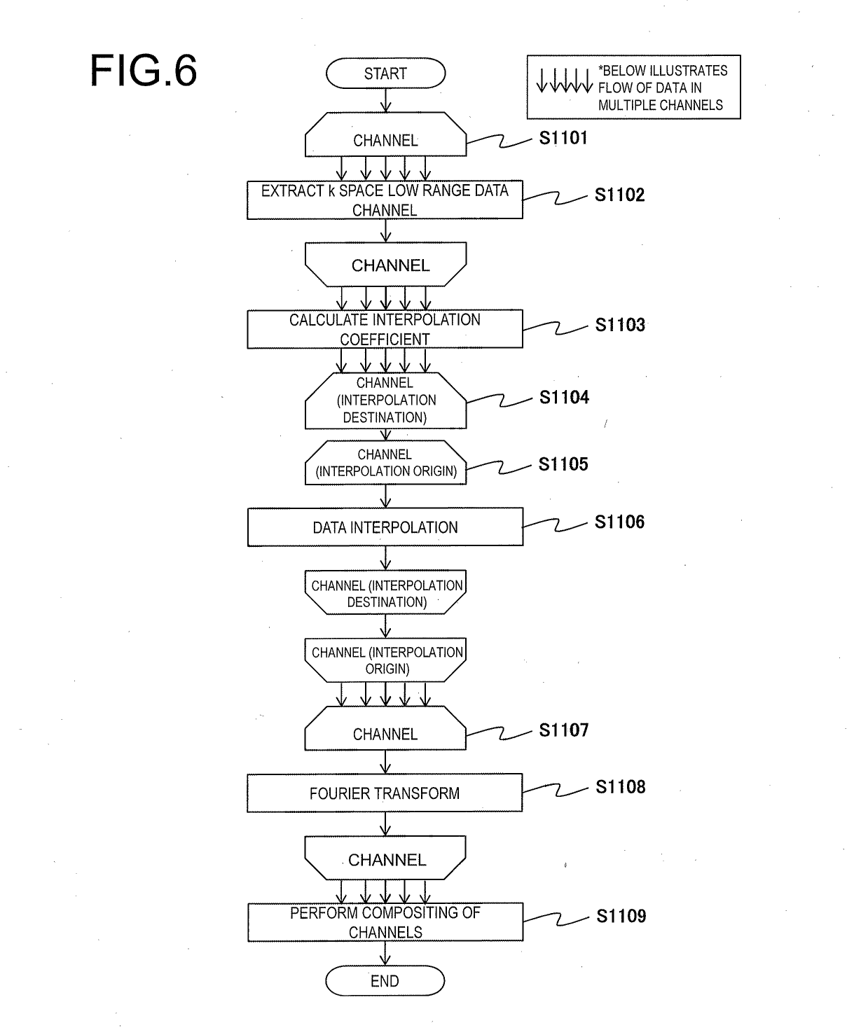

Here, for a comparison with respect to the flow of the image reconstruction processing performed by the image reconstruction section 220 of the present embodiment, the flow of the image reconstruction processing performed by the k space parallel imaging in the related art will be described with reference to FIG. 6. In FIG. 6, multiple arrows indicate the flows of data in multiple channels.

First, in each channel, from the acquired k space data, the k space low range data is extracted (Steps S1101 and S1102). The reason is that the interpolation coefficient is used in calculation, as described above.

Subsequently, the k space low range data extracted in Step S1102 is used such that the interpolation coefficient is calculated, as described above (Step S1103).

Subsequently, the interpolation processing using the interpolation coefficient obtained in Step S1103 is performed. Here, as described above, for each interpolation destination channel (Step S1104), all of the interpolation origin data is used (Step S1105), the interpolation data is generated (data interpolation) (Step S1106), and the k space is restored.

Succeedingly, the k space restored in Steps S1104, S1105, and S1106 is subjected to Fourier transform for each channel, and image data (channel image) of each channel is generated (Steps S1107 and S1108).

Lastly, compositing (channel compositing) of each channel image generated in Steps S1107 and S1108 is performed, and the reconstruction image is obtained (Step S1109). The compositing of the channels is performed by adopting sum-of-square compositing, for example, as described above.

[Image Reconstruction Processing Performed Through Interpolation Processing of Present Embodiment]

Subsequently, the interpolation processing section 222 of the present embodiment will be described. The interpolation processing section 222 of the present embodiment segments the interpolation data so as to generate element data for each piece of the interpolation origin data. That is, the interpolation processing section 222 divides the interpolation processing into two stages such as processing in which the interpolation coefficient is applied to the k space data as the interpolation origin data acquired in the corresponding channel, for each channel and the element data of the interpolation data of all of the channels is generated (element data generation processing), and processing in which the element data is added for each piece of the interpolation data (addition processing). The element data generation processing is executed in parallel in units of interpolation origin channels.

[Configuration of Interpolation Processing Section]

In order to realize the processing, as illustrated in FIG. 2, the interpolation processing section 222 of the present embodiment is provided with an element data generation section 223 which generates the element data by using the measured k space data of one channel and the interpolation coefficient, and an addition section 224 which adds the element data generated by the element data generation section.

The element data generation section 223 of the present embodiment applies the interpolation coefficient to the measured k space data for each channel and individually generates the element data of the interpolation data of all of the channels. That is, the k space data of the corresponding channel is used as the interpolation origin data for each interpolation origin channel, the element data of the interpolation data of the interpolation destination channel is generated. In this case, the element data of the interpolation data is generated with respect to all of the channels.

In addition, the addition section 224 individually adds the element data and obtains the interpolation data. The addition section 224 performs Fourier transform of the k space restored based on the interpolation data, thereby obtaining the channel image. That is, the elements of the interpolation data of all of the channels generated for each interpolation origin channel are added for each piece of the interpolation data, and the interpolation data is obtained. The k space of each channel restored based on the interpolation data is subjected to Fourier transform, and the channel image is obtained.

[Specific Example of Interpolation Processing of Present Embodiment]

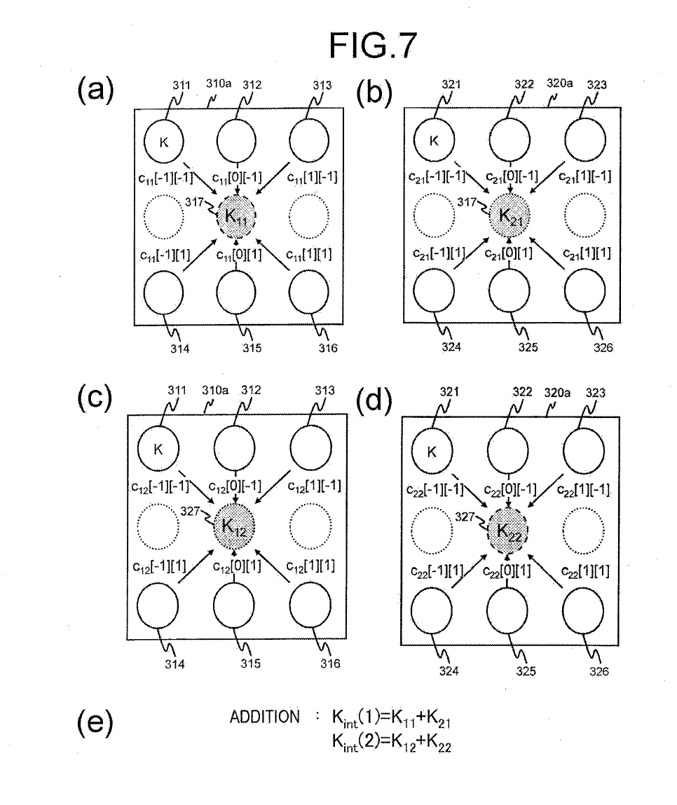

The interpolation processing performed by the interpolation processing section 222 of the present embodiment will be specifically described with reference to FIGS. 7(a) to 7(e). Here, for simplification, description will be given with reference to an example of a case where the number N of reception channels is 2.

Similar to FIGS. 3(c) and 5, FIGS. 7(a) to 7(d) schematically illustrate each of the enlarged small region 310a in the k space data 310 including the pixels 311 to 316 and 317 illustrated in FIG. 3(a), and the enlarged small region 320a in the k space data 320 including the pixels 321 to 326 and 327 illustrated in FIG. 3(b). The pixels 317 and 327 are pixels at the same pixel position. The definition of the interpolation coefficient cₘₙ[i][j] is similar to that in the method in the related art. In addition, the interpolation data of the channel n is expressed as K_{Int}(n).

In the method in the related art illustrated in FIG. 5, the interpolation data K_{Int}(1) of the channel 1 is generated through one computation by using the interpolation origin data of the channel 1 and the interpolation origin data of the channel 2. However, as illustrated in FIGS. 7 (a) to 7(e), the interpolation processing section 222 of the present embodiment generates each of the interpolation data K_{Int}(1) of the channel 1 and the interpolation data K_{Int}(2) of the channel 2 calculated through a computation divided into two stages (element data generation processing and addition processing).

The element data generation section 223 individually generates the element data Kₘₙ of the interpolation data Kit (n) of each channel n by using the interpolation origin data of the channel m. The process is performed with respect to all of the channels.

In the examples of FIGS. 7(a) to 7(d), as illustrated in FIGS. 7 (a) and 7 (c), the k space data of the pixels 311 to 316 of the channel 1 is used as the interpolation origin data such that the element data K₁₁ of the interpolation data of the pixel 317 of the channel 1 and the element data K₁₂ of the interpolation data of the pixel 327 of the channel 2 are generated. In addition, as illustrated in FIGS. 7 (b) and 7 (d), the k space data of the pixels 321 to 326 of the channel 2 is used as the interpolation origin data such that the element data K₂₁ of the interpolation data of the pixel 317 of the channel 1 and the element data K₂₂ of the interpolation data of the pixel 327 of the channel 2 are generated.

The addition section 224 adds the element Kₘₙ of the interpolation data of the channel n generated in each channel m, thereby generating the interpolation data K_{Int}(n) of the channel n.

As illustrated in FIG. 7 (e), the interpolation data K_{Int} (1) of the channel 1 is obtained by adding the element data K₁₁ of the pixel 317 generated in the channel 1, and the element data K₂₁ generated in the channel 2. In addition, the interpolation data K_{Int}(2) of the channel 2 is obtained by adding the element data K₁₂ of the pixel 327 generated in the channel 1 and the element data K₂₂ generated in the channel 2.

In this manner, in the interpolation processing of the present embodiment, since the element data can be generated by using only the data within the corresponding channel in each channel, processing can be performed in parallel for each channel.

[Suitability of Interpolation Processing of Present Embodiment]

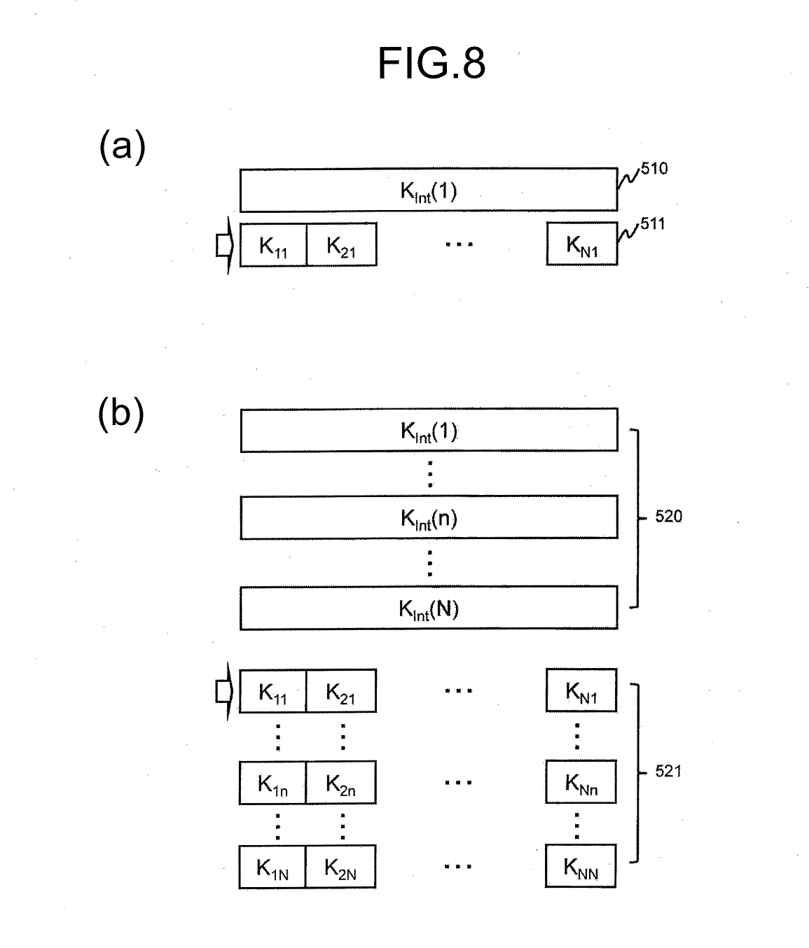

Here, suitability of the interpolation processing of the present embodiment will be described. FIGS. 8(a) and 8(b) schematically illustrate the interpolation processing in the k space parallel imaging.

As described above, when there are N channels, the interpolation data K_{Int} (1, kx, and ky) of the pixels (kx and ky) of the channel 1, generated through interpolation is expressed through the following Expression (11).

$$\begin{matrix}{K}_{1 nt} ( 1 , kx , ky ) = {c}_{11} [ - 1 ] [ - 1 ] \times K ( 1 , kx - 1 , ky - 1 ) + {c}_{11} [ 0 ] [ - 1 ] \times \\ K ( 1 , kx , ky - 1 ) + {c}_{11} [ 1 ] [ - 1 ] \times \\ K ( 1 , kx + 1 , ky - 1 ) + \\ {c}_{11} [ - 1 ] [ 1 ] \times K ( 1 , kx - 1 , ky + 1 ) + {c}_{11} [ 0 ] [ 1 ] \times \\ K ( 1 , kx , ky + 1 ) + {c}_{11} [ 1 ] [ 1 ] \times \\ K ( 1 , kx + 1 , ky + 1 ) + \\ {c}_{21} [ - 1 ] [ - 1 ] \times K ( 2 , kx - 1 , ky - 1 ) + {c}_{21} [ 0 ] [ - 1 ] \times \\ K ( 2 , kx , ky - 1 ) + {c}_{21} [ 1 ] [ - 1 ] \times \\ K ( 2 , kx + 1 , ky - 1 ) + \\ {c}_{21} [ - 1 ] [ 1 ] \times K ( 2 , kx - 1 , ky + 1 ) + {c}_{21} [ 0 ] [ 1 ] \times \\ K ( 2 , kx , ky + 1 ) + {c}_{21} [ 1 ] [ 1 ] \times \\ K ( 2 , kx + 1 , ky + 1 ) + \\ ⋮ \\ {c}_{N 1} [ - 1 ] [ - 1 ] \times K ( N , kx - 1 , ky - 1 ) + {c}_{N 1} [ 0 ] [ - 1 ] \times \\ K ( N , kx , ky - 1 ) + {c}_{N 1} [ 1 ] [ - 1 ] \times \\ K ( N , kx + 1 , ky - 1 ) + \\ {c}_{N 1} [ - 1 ] [ 1 ] \times K ( N , kx - 1 , ky + 1 ) + {c}_{N 1} [ 0 ] [ 1 ] \times \\ K ( N , kx , ky + 1 ) + {c}_{N 1} [ 1 ] [ 1 ] \times \\ K ( N , kx + 1 , ky + 1 ) \\ Here , \end{matrix} \\ \begin{matrix}{K}_{11} = {c}_{11} [ - 1 ] [ - 1 ] \times K ( 1 , kx - 1 , ky - 1 ) + {c}_{11} [ 0 ] [ - 1 ] \times \\ K ( 1 , kx , ky - 1 ) + {c}_{11} [ 1 ] [ - 1 ] \times \\ K ( 1 , kx + 1 , ky - 1 ) + \\ {c}_{11} [ - 1 ] [ 1 ] \times K ( 1 , kx - 1 , ky + 1 ) + {c}_{11} [ 0 ] [ 1 ] \times \\ K ( 1 , kx , ky + 1 ) + {c}_{11} [ 1 ] [ 1 ] \times \\ K ( 1 , kx + 1 , ky + 1 ) \\ {K}_{21} = {c}_{21} [ - 1 ] [ - 1 ] \times K ( 2 , kx - 1 , ky - 1 ) + {c}_{21} [ 0 ] [ - 1 ] \times \\ K ( 2 , kx , ky - 1 ) + {c}_{21} [ 1 ] [ - 1 ] \times \\ K ( 2 , kx + 1 , ky - 1 ) + \\ {c}_{21} [ - 1 ] [ 1 ] \times K ( 2 , kx - 1 , ky + 1 ) + {c}_{21} [ 0 ] [ 1 ] \times \\ K ( 2 , kx , ky + 1 ) + {c}_{21} [ 1 ] [ 1 ] \times \\ K ( 2 , kx + 1 , ky + 1 ) \\ … \\ {K}_{N 1} = {c}_{N 1} [ - 1 ] [ - 1 ] \times K ( N , kx - 1 , ky - 1 ) + {c}_{N 1} [ 0 ] [ - 1 ] \times \\ K ( 1 , kx , ky - 1 ) + {c}_{N 1} [ 1 ] [ - 1 ] \times \\ K ( 1 , kx + 1 , ky - 1 ) + \\ {c}_{N 1} [ - 1 ] [ 1 ] \times K ( 1 , kx - 1 , ky + 1 ) + {c}_{N 1} [ 0 ] [ 1 ] \times \\ K ( 1 , kx , ky + 1 ) + {c}_{N 1} [ 1 ] [ 1 ] \times \\ K ( 1 , kx + 1 , ky + 1 )\end{matrix} \qquad ( 11 )$$

In a case above, as illustrated in FIG. 8 (a), the interpolation data K_{Int}(1) of the channel 1 is expressed through the following Expression (12).

K_{Int}(1)=K₁₁+K₂₁+ and so on to +K_{N1} (12)

The above-referenced Expressions (11) and (12) express the processing in which the data interpolation is performed by using the data of all of the channels (N channels) as the interpolation origin data and the interpolation data K_{Int}(1) of the channel 1 is generated.

In this manner, processing 510 in which the interpolation data K_{Int}(1) of the channel 1 is generated can be considered as processing 511 in which processing of generating each of the elements K₁₁ to K_{N1} is combined.

FIG. 8(b) illustrates the extended application to all of the reception channels. Here, the element of the interpolation data of the channel n generated by using the data of the channel m as the interpolation origin data is Kₘₙ.

As illustrated in FIG. 8 (b), the interpolation data K_{Int}(n) of the channel n is expressed as the sum of each piece of the element data K₁₁ to K_{N1}. Therefore, the processing 520 generating the interpolation data of all of the channels K_{Int}(1) to K_{Int}(N) is expressed as the processing 521 in which the processing of generating each piece of the element data K₁₁ to K_{NN} is combined.

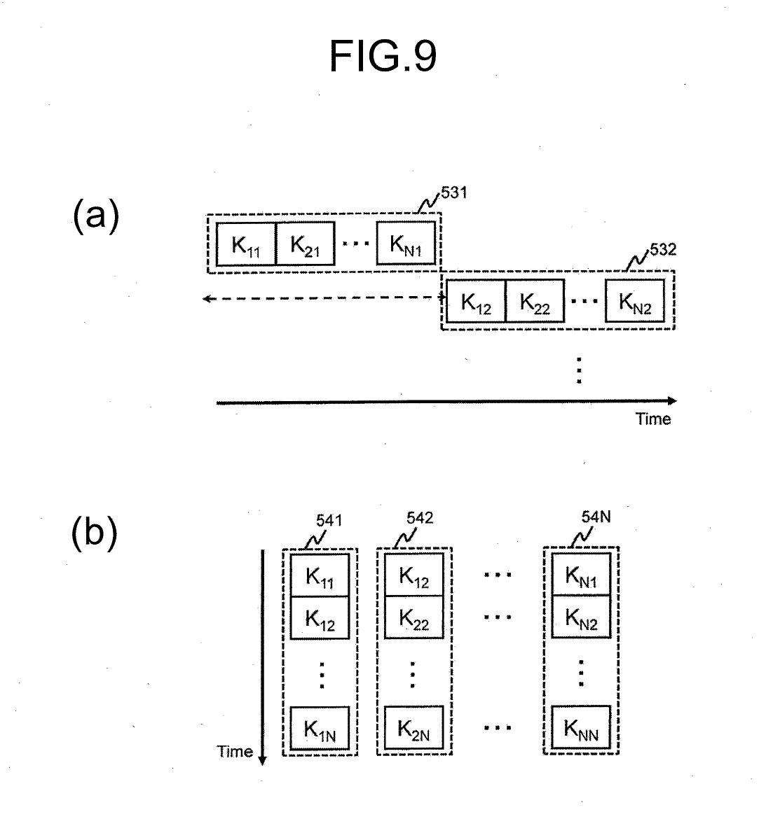

In the k space parallel imaging in the related art, the interpolation processing is performed in units of interpolation destination channels. However, in the interpolation processing of the present embodiment, a portion of the interpolation processing is performed in units of interpolation origin channels. Thereafter, the result is added for each interpolation destination channel. The difference with respect to the processing in the related art will be described with reference to FIGS. 9(a) and 9(b).

In the processing in the related art, as illustrated in FIG. 5, the interpolation data K_{Int}(1) of the channel 1 is generated by using the k space data of all of the channels as the interpolation origin data, and the interpolation data K_{Int}(2) of the channel 2 is generated by using the k space data of all of the channels. Lastly, the interpolation data K_{Int}(N) of the channel N is generated by using the data of all of the channels.

That is, as illustrated in FIG. 9(a), processes of generation processing of the interpolation data, such as generation processing 531 of the interpolation data K_{Int}(1) of the channel 1 and generation processing 532 of the interpolation data K_{Int}(2) of the channel 2 are repeated for each interpolation destination channel.

In each process of the generation processing 531 and 532 for each interpolation destination channel, the k space data of all of the channels is used as the interpolation origin data. Therefore, the interpolation origin data used in the processing between the processes of the generation processing 531 and 532 contend with each other. Therefore, the generation processing 532 cannot be executed during the processing of the generation processing 531. As a result thereof, each process of the generation processing has to be successively processed.

When the k space data of all of the channels is transferred to each process of the generation processing 531 and 532, each process of the generation processing can be executed in parallel. In this case, compared to a case where the processing is successively performed as described above, the memory corresponding to the multiplication of the number of channels is required to be ensured, and it is not realistic.

Meanwhile, in the present embodiment, as illustrated in FIG. 9(b), the interpolation processing is performed in units of interpolation origin channels (541 to 54N).

That is, in generation processing 541, in order to generate the element data KIN based on the element data Ku, only the k space data of the channel 1 is required as the interpolation origin data. The interpolation coefficient transferred to the generation processing 541 may only be C₁₁[i][j] to C_{1N}[i][j]. In addition, in generation processing 542, in order to generate the element data K_{2N} based on the element data K₂₁, only the k space data of the channel 2 is required as the interpolation origin data. The interpolation coefficient transferred to the generation processing 542 may only be C₂₁[i][j] to C_{2N}[i][j]. Similarly, in generation processing 54N, in order to generate the element data K_{NN} based on the element data K_{N1}, only the k space data of the channel N is required. The interpolation coefficient transferred to the generation processing 54N may only be C_{N1}[i][j] to C_{NN}[i][j].

In this manner, the pieces of data required in each process of the generation processing do not contend with each other. As a result, according to the technique of the present embodiment, each process of the generation processing can be executed in parallel with the memory having the same capacity as that in the related art, and thus, the processing time is shortened.

[Flow of Image Reconstruction Processing Performed Through Interpolation Processing of Present Embodiment]

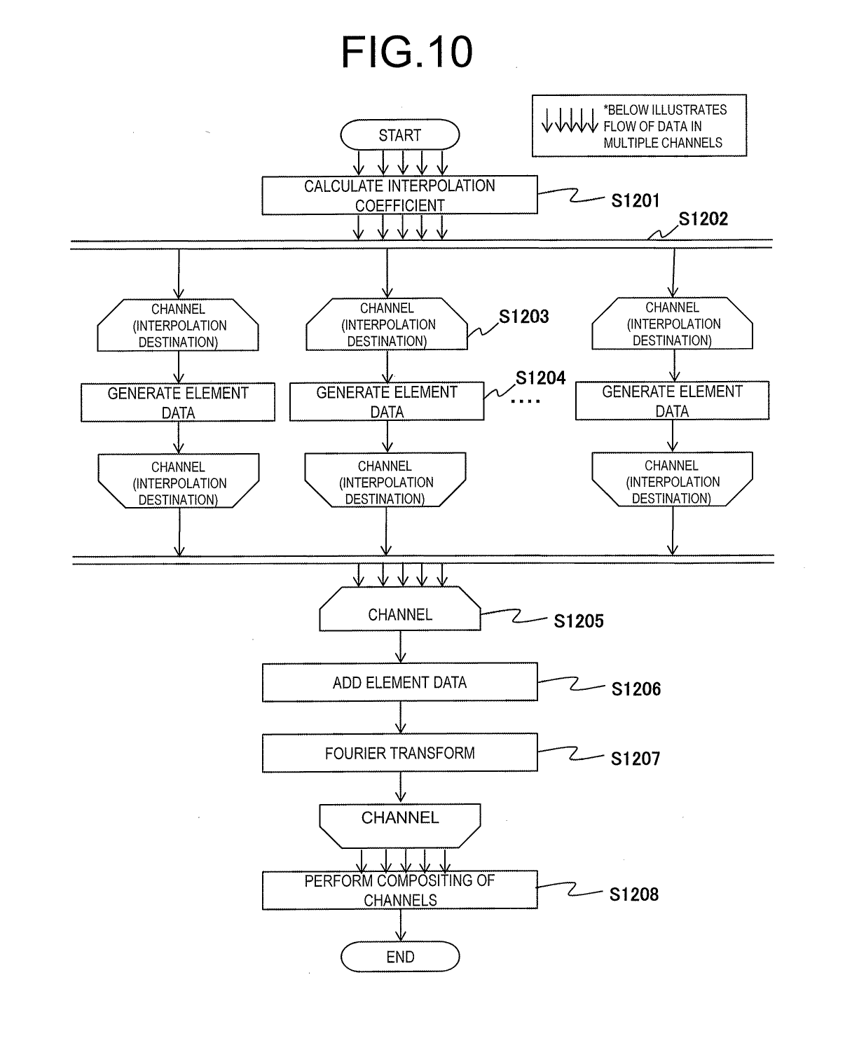

Subsequently, description will be given regarding a flow of the image reconstruction processing in which the interpolation processing is performed through the technique described above, which is performed by the image reconstruction section 220 of the present embodiment, and which is performed through the k space parallel imaging. FIG. 10 is a processing flow of the image reconstruction processing of the present embodiment.

First, the preprocessing section 221 extracts the k space low range data and calculates the interpolation coefficient (Step S1201). The calculation of the interpolation coefficient performed by extracting the k space low range data in order to calculate the interpolation coefficient and using the extracted k space low range data is the same as that in the related art.

When the interpolation coefficient is calculated, the element data generation section 223 of the present embodiment performs the element data generation processing in parallel for each interpolation origin channel (Step S1202).

In each process of the element data generation processing, the element data generation section 223 generates the element data of the interpolation data of the interpolation destination channel with respect to all of the interpolation destination channels (Step S1203, S1204).

Thereafter, for each channel (Step S1205), the addition section 224 adds the element data of each piece of the interpolation data (Step S1206), and the interpolation data is generated. The restored k space is subjected to Fourier transform (Step S1207), and the channel image is generated.

Lastly, the image compositing section 225 performs compositing of the channel images of each channel (Step S1208), and the reconstruction image is generated.

As described above, the MRI apparatus of the present embodiment includes the reception coil 161 that is provided with multiple channels, the measurement section 210 that performs thinning of the encoding step of the k space and measures the k space data for each of the channels, and the image reconstruction section 220 that applies a computation to the measured k space data and obtains the reconstruction image. The image reconstruction section 220 is provided with the preprocessing section 221 using the k space data so as to calculate a coefficient to be used in the computation, the interpolation processing section 222 executing the interpolation processing in which the coefficient is applied to the k space data and generating a channel image which is an image for each of the channels, and the image compositing section 225 performing compositing of the channel images and obtaining the reconstruction image. The interpolation processing section 222 is provided with the element data generation section 223 using the measured k space data of one of the channels and the coefficient so as to generate the element data of all of the channels, and the addition section 224 adding the element data generated by the element data generation section 223 for each of the channels. The element data generation section 223 generates the element data in parallel in units set in advance.

In this case, the interpolation processing may be processing in which the measured k space data is used and the interpolation data that is the thinned k space data is generated. The preprocessing section 221 may calculate the interpolation coefficient to be used in the interpolation processing, based on the measured k space data. The element data generation section 223 may apply the interpolation coefficient to the measured k space data of one of the channels and individually generates the element data of the interpolation data of all of the channels. The addition section 224 may individually add the element data for each of the channels, may obtain the interpolation data, and may perform Fourier transform of the k space restored based on the interpolation data, so as to obtain the channel image.

In addition, an image reconstruction method performed by the image reconstruction section 220 of the present embodiment includes an image reconstruction step of obtaining a reconstruction image from the k space data obtained by performing thinning of the encoding step of the k space and performing measurement in each of the reception coils 161 provided with multiple channels. The image reconstruction step includes a preprocessing step of using the k space data so as to calculate a coefficient to be used in the computation, an interpolation step of executing the interpolation processing in which the coefficient is applied to the k space data and generating a channel image which is an image for each of the channels, and an image compositing step of performing compositing of the channel images and obtaining the reconstruction image. The interpolation step includes an element data generation step of using the measured k space data of one of the channels and the coefficient such that the element data of all of the channels is generated in parallel in units set in advance, and an addition step of adding the generated element data for each of the channels.

In this case, the interpolation processing may be processing in which the measured k space data is used and the interpolation data that is the thinned k space data is generated. In the preprocessing step, the interpolation coefficient to be used in the interpolation processing may be calculated based on the measured k space data. In the element data generation step, the interpolation coefficient may be applied to the measured k space data of one of the channels and the element data of the interpolation data of all of the channels may be individually generated. In the addition step, the element data may be individually added for each of the channels, the interpolation data may be obtained, and Fourier transform of the k space restored based on the interpolation data may be performed such that the channel image is obtained.

In this manner, according to the present embodiment, the interpolation processing of the k space parallel imaging is segmented into two stages such as the element data generation processing in which the element data of the interpolation data is generated, and the addition processing in which the element data is added and the interpolation data is generated. The element data generation processing is segmented into multiple processing units and the processing is executed in parallel. In this case, the element data generation processing is segmented such that the data required in the processing does not contend with other processes of the segmented processing. For example, the element data generation processing is segmented into units of the interpolation origin channels and the processing is executed in parallel for each interpolation origin channel.

According to the present embodiment, during the interpolation processing, instead of executing the element data generation processing in parallel, the addition processing is performed. Therefore, compared to the technique in the related art, the process of the addition processing is increased. However, the processing quantity of the element data generation processing is greater than the addition processing. Therefore, according to the present embodiment, the effect of the paralleling in the element data generation processing exceeds the increase of the processing quantity caused due to the supplemented addition processing. Thus, it is possible to realize a state close to the ideal paralleling in processing, that is, the processing is increased in speed so as to correspond to the multiplication of the number of segmentations.

That is, according to the present embodiment, it is possible to avoid contention of data in the parallel processing and to enhance the efficiency of the paralleling. Thus, according to the present embodiment, compared to the processing in the related art, the efficiency of the paralleling is improved, and the reconstruction time can be shortened. Moreover, since the interpolation data which is ultimately obtained is completely the same as that of the technique in the related art, it is possible to obtain the same result as that of the processing in the related art without depending on an imaging sequence or the reception coil.

Therefore, according to the present embodiment, in the k space parallel imaging, the image reconstruction processing can be increased in speed without deteriorating the image quality.

### Modification Example 1

In the embodiment described above, when the element data generation processing is processed in parallel, the processing is segmented in units of interpolation origin channels. However, the unit of segmentation is not limited thereto. The element data generation section 223 may be configured to generate the element data in parallel in units of multiple channels set in advance.

The number of channels of the actually used reception coil 161 is often equal to or exceeding the ability of parallel computation provided in the control system 170. Therefore, for example, the unit of segmentation may be determined in accordance with the number of times of computation which can be processed in parallel by the CPU 171 in the control system 170.

For example, the control system 170 is provided with B substrates (B is an integer satisfying 0<B≦N, N the number of channels of the reception coil 161) as a computation section corresponding to the CPU 171. The B substrates can operate (execution of computation processing) in parallel. In this case, each of the substrates holds the k space data equal to or less than CEIL (N/B) channels and performs the element data generation processing by using the k space data. The factor CEIL(x) expresses the minimum integer equal to or greater than x. That is, when a number b (1≦b≦B) is applied to the substrate, the k space data held by the substrate b is k space data from (b−1)×CEIL(N/B)+1 channel to the channel having a smaller value between b×CEIL(N/B) and N.

In this modification example, the channel to be processed is allocated to each of the substrates, and the paralleling of the processing is performed in units of substrates. That is, the element data generation processing is segmented into B processes, and the parallel processing is performed.

For example, when the bth substrate holds k space data of e in a channel s, in the bth substrate, pieces of the element data Kₛ₁ to K_{sN}, K₍ₛ₊₁₎₁ to K_{(s+1)N}, and so on to Kₑ₁ to K_{eN} having the k space data of the channel to be held as the interpolation origin data are generated.

That is, in the bth substrate, regarding each of the channels from the channel s to the channel e, while having the k space data of the corresponding channel as the interpolation origin data, the element of the interpolation data of all of the channels is generated.

[Flow of Image Reconstruction Processing]

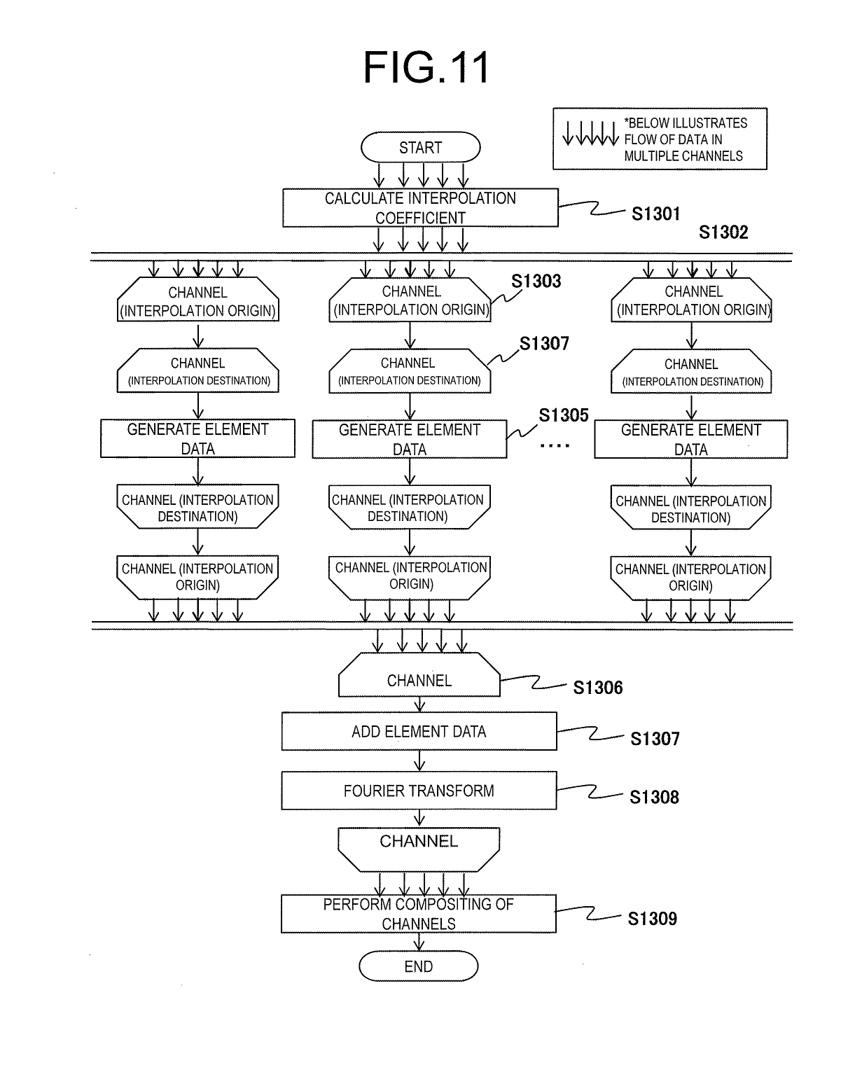

FIG. 11 illustrates a processing flow of the image reconstruction processing performed by the image reconstruction section 220 of this modification example through the k space parallel imaging.

First, the preprocessing section 221 extracts the k space low range data and calculates the interpolation coefficient (Step S1301).

When the interpolation coefficient is calculated, the element data generation section 223 performs the element data generation processing in parallel so as to individually generate the element data of each piece of the interpolation data of all of the channels regarding one or more interpolation origin channels allocated to the substrate in units of substrates (Step S1302 to S1305).

Thereafter, for each channel (Step S1306), the addition section 224 adds the element data of each piece of the interpolation data (Step S1307), and the interpolation data is generated. The restored k space is subjected to Fourier transform (Step S1308), and the channel image is generated.

Lastly, the image compositing section 225 performs compositing of the channel images of each channel (Step S1309), and the reconstruction image is generated.

### Modification Example 2

In the processing of the modification example described above, the element of the interpolation data generated in parallel is configured to be added after the parallel processing. However, the processing is not limited thereto. For example, as shown in the following Expression (13), the processing may be configured to perform addition regarding the channel to be processed in the substrate within each of the substrates, generate a second element data K_{bn}, and perform addition between the substrates thereafter.

$${K}_{bn} = {\sum }_{i = s}^{e} {K}_{i n} \qquad ( 13 )$$

In this case, the interpolation data K_{Int}(n) of the channel n in the addition processing of Step S1307 is calculated through the following Expression (14).

$${K}_{lnt} = {\sum }_{i = 1}^{B} {K}_{bn} \qquad ( 14 )$$

That is, the element data generation section 223 adds the generated element data in units of interpolation origin channels and transfers the result to the addition section 224. The addition section 224 adds the element data after being added in the element data generation section 223 in units of interpolation data, thereby generating the interpolation data.

In this manner, in each of the modification examples described above, the processing unit of the k space parallel imaging can be arbitrarily set. Therefore, the paralleling of the processing can be efficiently performed without depending on the configuration of the apparatus.

### Modification Example 3

In addition, as long as pieces of data do not contend with each other, the paralleling may be performed by segmenting the processing into equal to or more the number of channels. For example, the processing may be segmented into 2N (N is the number of reception channels) by segmenting one piece of channel data into two frequency encoding directions. In this case, the element data generation section 223 segments the k space data of each channel into pieces set in advance, thereby generating the element data in parallel in units of segmentations thereof.

### Second Embodiment

Subsequently, a second embodiment of the present invention will be described. In the present embodiment, the existing technology of increasing in speed is combined. Here, as the existing technology of increasing in speed, a technology of handling the interpolation processing of the k space to be transformed into processing in an image space (hereinafter, image space method) is used.

An MRI apparatus of the present embodiment basically has a configuration similar to the MRI apparatus 100 of the first embodiment. The functional block of the control system 170 of the present embodiment is also similar to that of the first embodiment. However, since the image space method is used, the processing of the preprocessing section 221 and the interpolation processing section 222 of the image reconstruction section 220 is different from that of the first embodiment. Hereinafter, regarding the present embodiment, description will be given while focusing on the configuration different from that of the first embodiment.

The interpolation processing of the image space method is processing in which aliasing is eliminated from an aliasing image obtained based on the measured k space data. Specifically, first, the aliasing image is generated based on the measured k space data of each channel. A result obtained by multiplying by a coefficient calculated in advance is added to each of the aliasing images of all of the channels, thereby obtaining an image from which aliasing of one channel is eliminated.

[Flow of Image Reconstruction Processing of Image Space Method]

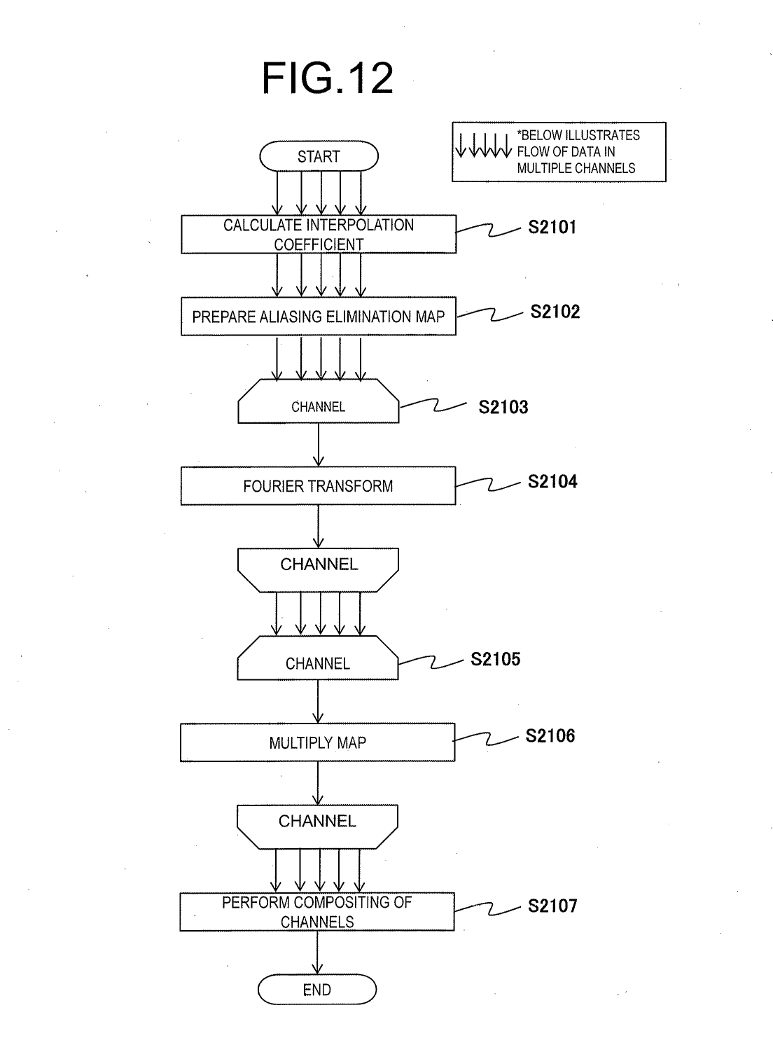

First, a general flow of the image reconstruction processing including aliasing elimination processing of the image space method will be described with reference to FIG. 12.

First, the low range data of the k space of each channel is extracted, and the interpolation coefficient is calculated (Step S2101). Extraction of data used in calculation of the interpolation coefficient and the calculation processing of the interpolation coefficient are similar to those of the first embodiment.

Subsequently, in the image space method, the calculated interpolation coefficient is transformed to an aliasing elimination map. That is, the aliasing elimination map is generated based on the interpolation coefficient (Step S3102). The aliasing elimination map is generated according to the procedure described below.

First, based on the following Expression (15), interpolation coefficients cₘₙ are respectively disposed at positions corresponding to k spaces kcₘₙ.

$${kc}_{mn} ( kx + i , ky + j ) = \{ \begin{matrix}{c}_{mn} [ i ] [ j ] & ( - 1 \leq i \leq 1 and j = \pm 1 ) \\ 1 & ( m = n and i = j = 0 ) \\ 0 & ( other than above )\end{matrix} \qquad ( 15 )$$

In accordance with the following Expression (16), Fourier transform is performed for each channel, and the aliasing elimination map is generated.

MAPₘₙ(x,y)=FT[kcₘₙ(kx+i,ky+j)] (16)

Here, the factor MAPₘₙ indicates the aliasing elimination map operated from the channel m to the channel n, and the factor FT indicates an operator to which Fourier transform is applied, respectively.

The aliasing elimination map operated from the channel m to the channel n is a map by which the aliasing image of the channel m is multiplied when aliasing of an image of the channel n is eliminated. Hereinafter, in the present embodiment, in this case, the channel m will be referred to as the interpolation origin channel, and the channel n will be referred to as the interpolation destination channel.

Subsequently, in the image space method, for each channel (Step S2103), the k space data which is thinned and measured in the corresponding channel is subjected to Fourier transform, and the aliasing image is generated (Step S2104). For example, in a case of the channel n, an aliasing image FT [K(n, kx, and ky)] is obtained based on a k space data K (n, kx, and ky) which is thinned and measured. The aliasing image is generated as many as the number N of channels.

Succeedingly, as shown in Expression (17) Expression, the aliasing image FT [K(m, kx, and ky)] of each of the interpolation origin channels m is multiplied by the aliasing elimination map MAPₘₙ (x and y) from the channel m to the channel n. The multiplied results of all of the interpolation origin channels are added, thereby generating an image Iₙ (x and y) of the interpolation destination channel n from which aliasing is eliminated (Step S2106). The process is performed with respect to each of the interpolation destination channels (Step S2105). The image from which aliasing is eliminated is a channel image.

$$\begin{matrix}{I}_{n} ( x , y ) = {MAP}_{1 n} ( x , y ) \times FT [ K ( 1 , kx , ky ) ] + \\ {MAP}_{2 n} ( x , y ) \times FT [ K ( 2 , kx , ky ) ] + \\ ⋮ \\ {MAP}_{Nn} ( x , y ) \times FT [ K ( N , kx , ky ) ] \\ = {I}_{1 n} + {I}_{2 n} + ⋯ + {I}_{Nn}\end{matrix} \qquad ( 17 )$$

Lastly, compositing of the images from which aliasing is eliminated in each channel (channel images) is performed so as to obtain a result image (Step S2107).

In this manner, in the image space method, there is no need to repeat the processing regarding all of the thinned pixels in the k space in order to transform a convolution computation to a map multiplication. However, there is a need to generate aliasing elimination images I₁₁ to I_{NN} operated from each of the interpolation origin channels to each of the interpolation destination channels.

[Image Reconstruction Processing Performed Through Interpolation Processing of Present Embodiment]

In the present embodiment, the image space method is combined with the technique described in the first embodiment, and the paralleling of the generation processing of the aliasing elimination image is performed.

The interpolation processing section 222 of the present embodiment segments the channel image so as to generate the element of the interpolation origin channel. That is, for each interpolation origin channel, the interpolation processing section 222 of the present embodiment divides the interpolation processing into two stages such as the element data generation processing in which the aliasing image obtained by reconstructing the k space data acquired in the corresponding channel is multiplied by the calculated aliasing elimination map and the element data of the channel image is generated regarding all of the channels, and the addition processing in which the element data is added for each channel. The element data generation processing is executed in parallel in units of interpolation origin channels.

First, the preprocessing section 221 of the present embodiment calculates the interpolation coefficient based on the measured k space data and generates the aliasing elimination map operated from the interpolation origin channel to the interpolation destination channel regarding each channel based on the calculated interpolation coefficient. The aliasing elimination map is calculated through a technique similar to that in the related art.

The element data generation section 223 of the present embodiment multiplies the aliasing image for each interpolation origin channel by the aliasing elimination map, thereby individually generating the element data of the channel image after aliasing is eliminated from all of the channels.

That is, for each of the interpolation origin channels m, by using the aliasing image obtained based on the k space data of the corresponding channel m as the interpolation origin data, the aliasing elimination map operated from each of the interpolation origin channels m to the interpolation destination channel n is individually multiplied. Therefore, the element data of the aliasing elimination image of each of the interpolation destination channels is generated.

When the interpolation origin channel is m, the element data of the aliasing elimination image of each of the interpolation destination channels generated herein is Iₘ₁=MAPₘ₁(x and y)×FT [K(m, kx, and ky)], Iₘ₂=MAPₘ₂(x and y)×FT [K(m, kx, and ky)], and so on to I_{mN}=MAP_{mN} (x and y)×FT [K(m, kx, and ky)] obtained by multiplying the aliasing image FT [K(m, kx, and ky)] of the interpolation origin channel m by the aliasing elimination map operated from the interpolation origin channel m to each of the interpolation destination channels (1 to N).

The addition section 224 individually adds the element data and obtains the channel image. In the present embodiment, the addition section 224 adds the processing result obtained by the element data generation section 223 and obtains the channel image which is the image for each channel. In the present embodiment, the element data of the aliasing elimination image of each of the interpolation destination channels generated for each interpolation origin channel is added for each interpolation destination, and the aliasing elimination image of the interpolation destination channel is obtained as the channel image.

For example, when the interpolation destination channel is n, the aliasing elimination image Iₙ(x and y) of the interpolation destination channel n is obtained through the following Expression (18).

Iₙ(x and y)=I₁ₙ+I₂ₙ+ and so on to +I_{Nn} (18)

Similar to the first embodiment, the image compositing section 225 performs compositing of the channel images of each channel, thereby obtaining the reconstruction image. The technique of image compositing is similar to that of the first embodiment.

[Flow of Image Reconstruction Processing Performed Through Interpolation Processing of Present Embodiment]

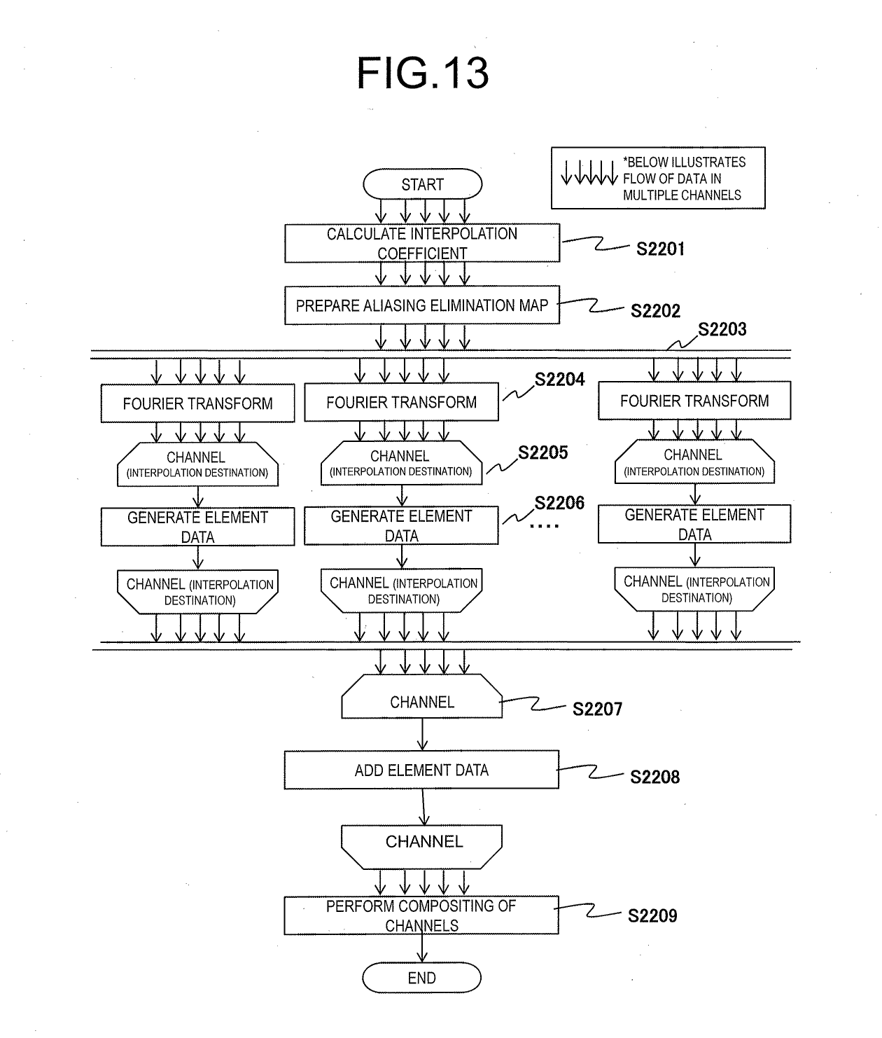

Subsequently, description will be given with reference to FIG. 13 regarding a flow of the image reconstruction processing performed by the image reconstruction section 220 of the present embodiment through the image space method.

The preprocessing section 221 of the present embodiment calculates the interpolation coefficient through a technique similar to that of the first embodiment (Step S2201). Through a technique similar to the technique in the related art, the aliasing elimination map is generated by using the above-referenced Expression (16) (Step S2202).

When the aliasing elimination map is calculated, the element data generation section 223 of the present embodiment generates the element data of the aliasing elimination image of the interpolation destination channel regarding all of the interpolation destination channels. In the present embodiment, the element data generation section 223 executes the below-described processing of Steps S2204 to S2206 in parallel in units of interpolation origin channels (Step S2203).

Step S2204: The k space data of the interpolation origin channel is subjected to Fourier transform, and the aliasing image is obtained.

Steps S2205 and S2206: The aliasing image is individually multiplied by the aliasing elimination map operated from the corresponding channel to each channel, and regarding all of the channels, the element data of the aliasing elimination image for each interpolation destination channel is generated.

Thereafter, for each channel (Step S2207), the addition section 224 adds the element data of each of the aliasing elimination images (Step S2208), thereby generating the channel image.

Lastly, the image compositing section 225 performs compositing of the channel images of each channel (Step S2209), thereby generating the reconstruction image.

In the present embodiment as well, the unit for performing processing in parallel is not limited to the unit of one channel. That is, each of the modification examples of the first embodiment can also be applied to the present embodiment.

As described above, similar to the first embodiment, the MRI apparatus of the present embodiment includes the reception coil 161 provided with multiple channels, the measurement section 210, and the image reconstruction section 220. The image reconstruction section 220 is provided with the preprocessing section 221, the interpolation processing section 222, and the image compositing section 225. The interpolation processing section 222 is provided with the element data generation section 223 and the addition section 224. The element data generation section 223 generates the element data in parallel in units set in advance.

In this case, the interpolation processing may be processing in which aliasing is eliminated from the aliasing image obtained based on the measured k space data. The preprocessing section 221 may generate the elimination map for eliminating aliasing from the measured k space data. The element data generation section 223 may multiply the aliasing image of one of the channels by the elimination map so as to individually generate the element data of the channel image after aliasing is eliminated from all of the channels. The addition section 224 may individually add the element data for each of the channels so as to obtain the channel image.

In addition, similar to the first embodiment, the image reconstruction method performed by the image reconstruction section 220 of the present embodiment includes the image reconstruction step. The image reconstruction step includes the preprocessing step, the interpolation step, and the image compositing step. The interpolation step includes the element data generation step of executing the processing in parallel in units set in advance, and the addition step.

In this case, the interpolation processing is processing in which aliasing is eliminated from the aliasing image obtained based on the measured k space data. In the preprocessing step, the elimination map for eliminating aliasing from the measured k space data may be generated. In the element data generation step, the aliasing image of one of the channels may be multiplied by the elimination map such that the element data of the channel image after aliasing is eliminated from all of the channels may be individually generated. In the addition step, the element data may be individually added for each of the channels such that the channel image is obtained.

In this manner, according to the present embodiment, the interpolation processing is segmented into two stages such as the element data generation processing in which the element data of the aliasing elimination image is generated, and the addition processing in which the element data is added and the aliasing elimination image is obtained. The element data generation processing is segmented into multiple processing units and the processing is executed in parallel. In this case, the element data generation processing is segmented such that the data required in the processing does not contend with other processes of the segmented processing. For example, the element data generation processing is segmented into units of the interpolation origin channels and the processing is executed in parallel for each interpolation origin channel.

In the present embodiment as well, in each process of the parallel processing, there is no contention of data. Therefore, similar to the first embodiment, the efficiency of the paralleling of processing is improved, and the reconstruction time can be shortened. Moreover, the image ultimately obtained after aliasing is eliminated for each channel is completely the same as that obtained through the technique in the related art. Therefore, it is possible to obtain the same result as that of the processing in the related art without depending on the imaging sequence or the reception coil.

In this manner, according to the present embodiment, even in a case where the image space method is used as the technology of increasing in speed, efficient processing paralleling can be performed.

In the embodiment described above, description has been given with reference to an example of a case where the image space method is used as the technology of increasing in speed. However, the embodiment is not limited thereto. Even in a case of other technologies of increasing in speed, when the processing can be segmented under the similar concept, the parallel processing can be applied by combining the technique of the first embodiment.

As other methods of increasing in speed, for example, there is a DVC method disclosed in the specification of US. Patent Application Publication No. 2010/0244825. In the DVC method, interpolation of the k space of each channel and compositing of the channels thereof are performed at the same time. The compositing of the channels is performed in the k space.

### Third Embodiment

Subsequently, a third embodiment of the present invention will be described. In the present embodiment, the paralleling described in the first and second embodiments is applied to the processing in a hybrid space.

An MRI apparatus of the present embodiment basically has a configuration similar to the MRI apparatus 100 of the first embodiment or the second embodiment. The functional block of the control system 170 of the present embodiment is also similar to that of the first embodiment. However, since the space for performing the interpolation processing is different, the processing of the preprocessing section 221 and the interpolation processing section 222 of the image reconstruction section 220 is different from that of the second embodiment. Hereinafter, regarding the present embodiment, description will be given while focusing on the configuration different from that of the second embodiment.

In the second embodiment described above, the processing is increased in speed by performing the computation of the k space through the transform to an image space and utilizing a convolution-type computation which is multiplied via Fourier trans form.

In Expression (10) indicating the interpolation processing of the k space data and Expression (17) indicating the multiplication of the image data, there is a relationship in which both the sides are subjected to two-dimension Fourier transform. Since even in any of the spaces, the computation of the interpolation processing is established. Therefore, even in a form in the middle of transform from Expression (10) to Expression (17), for example, even in the hybrid space of a stage subjected to one-dimensional Fourier transform (for example, only the kx-direction), the computation of the interpolation processing is established.

The interpolation processing section 222 of the present embodiment executes the interpolation processing in the hybrid space obtained by performing one-dimensional Fourier transform of the measured k space data. That is, hybrid space data obtained by performing one-dimensional Fourier transform of the measured k space data is interpolated.

Hereinafter, in the present embodiment, description will be given with reference to an example of a case of performing the interpolation processing based on x-ky space data (hybrid space data) obtained by performing Fourier transform in only the kx-direction of the k space data.

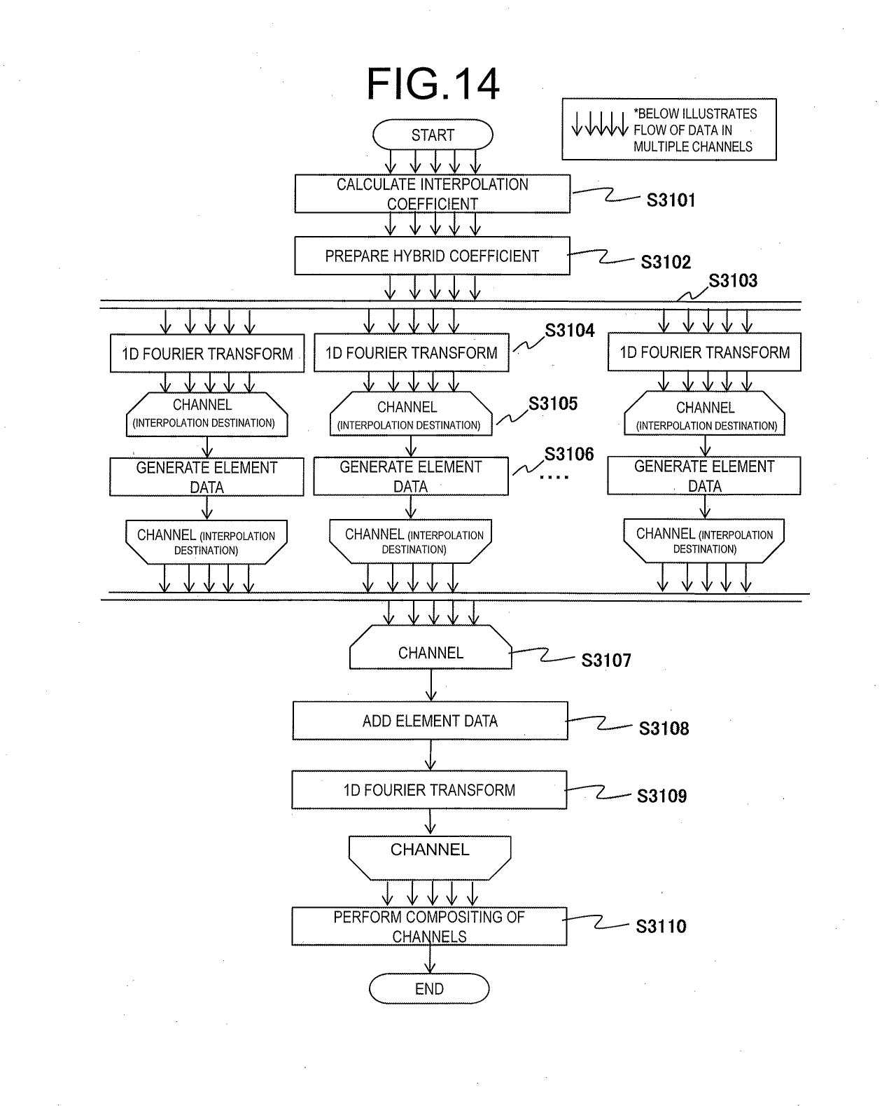

Hereinafter, the processing of each section of the present embodiment will be described in accordance with the processing flow of FIG. 14.

The preprocessing section 221 generates a hybrid coefficient for interpolating the hybrid space data based on the measured k space data as a coefficient to be used in the interpolation processing. Here, first, the interpolation coefficient is calculated (Step S3101). Then, the hybrid coefficient is generated based on the calculated interpolation coefficient (Step S3102).

The hybrid coefficient is generated by performing one-dimensional Fourier transform of the above-referenced Expression (15) obtained by disposing each of the interpolation coefficients Cₘₙ at a position corresponding to the k space kcₘₙ. That is, the preprocessing section 221 calculates the hybrid coefficient through the following Expression (19).

Hybridₘₙ(x,ky)=FT×[kcₘₙ(kx+i,ky+j)] (19)

Here, the factor Hybridₘₙ indicates the hybrid coefficient operated from the channel m to the channel n, and the factor FTx indicates an operator of applying Fourier transform in the x-direction, respectively.

The hybrid coefficient operated from the channel m to the channel n is a coefficient by which the hybrid space data of the channel m is multiplied when the hybrid space data of the channel n is interpolated. Hereinafter, in the present embodiment, in this case, the channel m will be referred to as the interpolation origin channel, and the channel n will be referred to as the interpolation destination channel.

Subsequently, the element data generation section 223 applies the hybrid coefficient to the hybrid space data for each interpolation origin channel and performs the element data generation processing of individually generating the element data of the hybrid space data after all of the channels are interpolated.

Specifically, the element data generation section 223 of the present embodiment executes the below-described element data generation processing in parallel (Step S3103).

Step S3104: The k space data of the corresponding channel which is thinned and measured is subjected to one-dimensional Fourier transform (Step S3104), thereby calculating hybrid data.

Steps S3105 and S3106: The hybrid coefficient which operates to each channel from the corresponding channel is applied to the hybrid data, and the element data of the hybrid space data after the interpolation for each interpolation destination channel is obtained. In this case, multiplication and addition are performed in the x-direction after Fourier transform is applied, and the convolution computation is performed in the ky-direction in which Fourier transform is not applied.

For each channel (Step S3107), the addition section 224 adds the element data (Step S3108), one-dimensional Fourier transform is performed in the y-direction (Step S3109), and the channel image is generated.

Lastly, the image compositing section 225 performs compositing of the channel images of each channel (Step S3109), thereby generating the reconstruction image.

In the present embodiment, description has been given with reference to an example of a case where the interpolation processing is executed in the hybrid space which is subjected to one-dimensional Fourier transform in the kx-direction. However, the hybrid space for executing the interpolation processing may be a hybrid space which is subjected to one-dimensional Fourier transform in the ky-direction.

In addition, in the present embodiment as well, the unit for performing processing in parallel is not limited to the unit of one channel. That is, each of the modification examples of the first embodiment can also be applied to the present embodiment.

As described above, similar to the first embodiment, the MRI apparatus of the present embodiment includes the reception coil 161 provided with multiple channels, the measurement section 210, and the image reconstruction section 220. The image reconstruction section 220 is provided with the preprocessing section 221, the interpolation processing section 222, and the image compositing section 225. The interpolation processing section 222 is provided with the element data generation section 223 and the addition section 224. The element data generation section 223 generates the element data in parallel in units set in advance.

In this case, the interpolation processing may be processing of interpolating the hybrid space data obtained by performing one-dimensional Fourier transform of the measured k space data. The preprocessing section 221 may generate the hybrid coefficient for interpolating the hybrid space data based on the measured k space data. The element data generation section 223 may apply the hybrid coefficient to the hybrid space data of one of the channels and may individually generate the element data of the hybrid space data after all of the channels are interpolated. The addition section 224 may individually add the element data for each of the channels and one-dimensional Fourier transform of the result of the addition may be performed so as to obtain the channel image.

In addition, similar to the first embodiment, the image reconstruction method performed by the image reconstruction section 220 of the present embodiment includes the image reconstruction step. The image reconstruction step includes the preprocessing step, the interpolation step, and the image compositing step. The interpolation step includes the element data generation step of executing the processing in parallel in units set in advance, and the addition step.

In this case, the interpolation processing may be processing of interpolating the hybrid space data obtained by performing one-dimensional Fourier transform of the measured k space data. In the preprocessing section step, the hybrid coefficient for interpolating the hybrid space data may be generated based on the measured k space data. In the element data generation step, the hybrid coefficient may be applied to the hybrid space data of one of the channels and the element data of the hybrid space data after all of the channels are interpolated may be individually generated. In the addition step, the element data may be individually added for each of the channels and one-dimensional Fourier transform of the result of the addition may be performed such that the channel image is obtained.

In this manner, according to the present embodiment, the interpolation processing is segmented into two stages such as the element data generation processing in which the element data of the hybrid space data after the interpolation is generated, and the addition treatment in which the element data is added and the aliasing elimination image is obtained. The element data generation processing is segmented into multiple processing units and the processing is executed in parallel. In this case, in this case, the element data generation processing is segmented such that the data required in the processing does not contend with other processes of the segmented processing. For example, the element data generation processing is segmented into units of the interpolation origin channels and the processing is executed in parallel for each interpolation origin channel.

In the present embodiment as well, in each process of the parallel processing, there is no contention of data. Therefore, similar to the first embodiment, the efficiency of the paralleling of processing is improved, and the reconstruction time can be shortened. Moreover, the hybrid space data ultimately obtained after the interpolation for each channel is completely the same as that obtained through the technique in the related art. Therefore, it is possible to obtain the same result as that of the processing in the related art without depending on the imaging sequence or the reception coil.

In each of the embodiments and each of the modification examples, the image reconstruction section 220 is described to be realized by the control system 170 provided in the MRI apparatus 100. However, the image reconstruction section 220 is not limited thereto. For example, the image reconstruction section 220 may be configured to realize all or a portion of the function on an information processing device or the like which can transmit and receive data with respect to the MRI apparatus 100 and is independent from the MRI apparatus 100.

Moreover, in each of the embodiments and each of the modification examples, in the configuration of the control system 170 which realizes the parallel processing, the number and the type thereof are not concerned as long as the configuration can independently execute the processing, such as a CPU (core or thread), a substrate (GPU, dedicated board, or the like), a PC, a server, a cloud PC. In addition, as the number of segmentations of the processing when the processing is performed in parallel, an optimal value may be empirically determined based on the general increase in speed due to paralleling, and the general cost.

The embodiment of the present invention is not limited to each of the embodiments described above, and various additions, changes, and the like can be made without departing from the gist of the invention.

### REFERENCE SIGNS LIST

100 MRI APPARATUS, 101 OBJECT, 120 STATIC MAGNETIC FIELD GENERATION SYSTEM, 130 GRADIENT MAGNETIC FIELD GENERATION SYSTEM, 131 GRADIENT MAGNETIC FIELD COIL, 132 GRADIENT MAGNETIC FIELD POWER SUPPLY, 140 SEQUENCER, 150 TRANSMISSION SYSTEM, 151 TRANSMISSION COIL, 152 TRANSMISSION PROCESSING SECTION, 160 RECEPTION SYSTEM, 161 RECEPTION COIL, 162 RECEPTION PROCESSING SECTION, 170 CONTROL SYSTEM, 171 CPU, 172 STORAGE DEVICE, 173 DISPLAY DEVICE, 174 INPUT DEVICE, 210 MEASUREMENT SECTION, 220 IMAGE RECONSTRUCTION SECTION, 221 PREPROCESSING SECTION, 222 INTERPOLATION PROCESSING SECTION, 223 ELEMENT DATA GENERATION SECTION, 224 ADDITION SECTION, 225 IMAGE COMPOSITING SECTION, 300 k SPACE LOW RANGE DATA, 300a SMALL REGION AS PORTION OF k SPACE LOW RANGE DATA, 301 ADJACENT PIXEL, 302 ADJACENT PIXEL, 303 ADJACENT PIXEL, 304 ADJACENT PIXEL, 305 ADJACENT PIXEL, 306 ADJACENT PIXEL, 307, INTERPOLATION TARGET PIXEL, 310 k SPACE DATA, 310a SMALL. REGION AS PORTION OF k SPACE LOW RANGE DATA, 311 ADJACENT PIXEL, 312 ADJACENT PIXEL, 313 ADJACENT PIXEL, 314 ADJACENT PIXEL, 315 ADJACENT PIXEL, 316 ADJACENT PIXEL, 317 INTERPOLATION TARGET PIXEL, 320 k SPACE DATA, 320a SMALL REGION AS PORTION OF k SPACE LOW RANGE DATA, 321 ADJACENT PIXEL, 322 ADJACENT PIXEL, 323 ADJACENT PIXEL, 324 ADJACENT PIXEL, 325 ADJACENT PIXEL, 326 ADJACENT PIXEL, 327 INTERPOLATION TARGET PIXEL, 510 INTERPOLATION DATA GENERATION PROCESSING, 511 INTERPOLATION DATA ELEMENT GENERATION PROCESSING, 520 INTERPOLATION DATA GENERATION PROCESSING, 521 INTERPOLATION DATA ELEMENT GENERATION PROCESSING, 531 INTERPOLATION DATA GENERATION PROCESSING OF CHANNEL 1, 532 INTERPOLATION DATA GENERATION PROCESSING OF CHANNEL 2, 541 INTERPOLATION DATA ELEMENT GENERATION PROCESSING, 542 INTERPOLATION DATA ELEMENT GENERATION PROCESSING, 54N INTERPOLATION DATA ELEMENT GENERATION PROCESSING

1. A magnetic resonance imaging apparatus comprising:
a reception coil that is provided with multiple channels;
a measurement section that performs thinning of an encoding step of a k space and measures k space data for each of the channels; and
an image reconstruction section that applies a computation to the measured k space data and obtains a reconstruction image,
wherein the image reconstruction section is provided with a preprocessing section using the k space data so as to calculate a coefficient to be used in the computation, an interpolation processing section executing interpolation processing in which the coefficient is applied to the k space data and generating a channel image which is an image for each of the channels, and an image compositing section performing compositing of the channel images and obtaining the reconstruction image,
wherein the interpolation processing section is provided with an element data generation section using the measured k space data of one of the channels and the coefficient so as to generate element data of all of the channels, and an addition section adding the element data generated by the element data generation section for each of the channels, and
wherein the element data generation section generates the element data in parallel in units set in advance.

2. The magnetic resonance imaging apparatus according to claim 1,
wherein the interpolation processing is processing in which the measured k space data is used and interpolation data that is the thinned k space data is generated,
wherein the preprocessing section calculates an interpolation coefficient to be used in the interpolation processing, based on the measured k space data,
wherein the element data generation section applies the interpolation coefficient to the measured k space data of one of the channels and individually generates the element data of the interpolation data of all of the channels, and
wherein the addition section individually adds the element data for each of the channels, obtains the interpolation data, and performs Fourier transform with respect to the k space data which is restored based on the interpolation data, so as to obtain the channel image.

3. The magnetic resonance imaging apparatus according to claim 1,
wherein the interpolation processing is processing in which aliasing of an aliasing image obtained from the measured k space data is eliminated,
wherein the preprocessing step generates an elimination map for eliminating aliasing from the measured k space data,
wherein the element data generation section multiplies the aliasing image of one of the channels by the elimination map so as to individually generate the element data of the channel image after aliasing is eliminated from all of the channels, and
wherein the addition section individually adds the element data for each of the channels so as to obtain the channel image.

4. The magnetic resonance imaging apparatus according to claim 1,
wherein the interpolation processing is processing in which hybrid space data obtained by performing one-dimensional Fourier transform with respect to the measured k space data is interpolated,
wherein the preprocessing section generates a hybrid coefficient interpolating the hybrid space data, based on the measured k space data,
wherein the element data generation section applies the hybrid coefficient to the hybrid space data of one of the channels and individually generates the element data of the hybrid space data after all of the channels are interpolated, and
wherein the addition section individually adds the element data for each of the channels and obtains the channel image by performing one-dimensional Fourier transform with respect to a result of the addition.

5. The magnetic resonance imaging apparatus according to claim 1,
wherein the element data generation section generates the element data in parallel in each unit of channel.

6. The magnetic resonance imaging apparatus according to claim 1,
wherein the element data generation section generates the element data in parallel in units of multiple channels set in advance.

7. The magnetic resonance imaging apparatus according to claim 6, further comprising:
a control section that performs processing of computations in parallel,
wherein a unit of generation performed in parallel is set in accordance with the number of times of computation which can be processed in parallel by the control section.

8. The magnetic resonance imaging apparatus according to claim 6,
wherein the element data generation section adds the generated element data in units of channels, and
wherein the addition section adds the element data after addition in the element data generation section.

9. The magnetic resonance imaging apparatus according to claim 1,
wherein the element data generation section segments the k space data of each channel into pieces set in advance and generates the element data in parallel in units of segmentations.

10. An image reconstruction method in a magnetic resonance imaging apparatus, comprising:
an image reconstruction step of applying a computation to k space data obtained by performing thinning of an encoding step of a k space and performing measurement, and obtaining a reconstruction image in each of reception coils provided with multiple channels,
wherein the image reconstruction step includes a preprocessing step of using the k space data so as to calculate a coefficient to be used in the computation, an interpolation step of executing interpolation processing in which the coefficient is applied to the k space data and generating a channel image which is an image for each of the channels, and an image compositing step of performing compositing of the channel images and obtaining the reconstruction image, and
wherein the interpolation step includes an element data generation step of using the measured k space data of one of the channels and the coefficient such that element data of all of the channels is generated in parallel in units set in advance, and an addition step of adding the generated element data for each of the channels.

11. The image reconstruction method according to claim 10,
wherein the interpolation processing is processing in which the measured k space data is used and interpolation data that is the thinned k space data is generated,
wherein in the preprocessing step, an interpolation coefficient to be used in the interpolation processing is calculated based on the measured k space data,
wherein in the element data generation step, the interpolation coefficient is applied to the measured k space data of one of the channels and the element data of the interpolation data of all of the channels is individually generated, and
wherein in the addition step, the element data is individually added for each of the channels, the interpolation data is obtained, and Fourier transform is performed with respect to the k space data which is restored based on the interpolation data, such that the channel image is obtained.

12. The image reconstruction method according to claim 10,
wherein the interpolation processing is processing in which aliasing is eliminated from an aliasing image obtained from the measured k space data,
wherein in the preprocessing step, an elimination map for eliminating aliasing from the measured k space data is generated,
wherein in the element data generation step, the aliasing image of one of the channels is multiplied by the elimination map such that the element data of the channel image after aliasing is eliminated from all of the channels is individually generated, and
wherein in the addition step, the element data for each of the channels is individually added such that the channel image is obtained.

13. The image reconstruction method according to claim 10,
wherein the interpolation processing is processing in which hybrid space data obtained by performing one-dimensional Fourier transform with respect to the measured k space data is interpolated,
wherein in the preprocessing section step, a hybrid coefficient interpolating the hybrid space data is generated based on the measured k space data,
wherein in the element data generation step, the hybrid coefficient is applied to the hybrid space data of one of the channels and the element data of the hybrid space data after all of the channels are interpolated is individually generated, and
wherein in the addition step, the element data is individually added for each of the channels and the channel image is obtained by performing one-dimensional Fourier transform with respect to a result of the addition.
